(19) United States
(12) Patent Application Publication
(10) Pub. No.: US 2024/0122987 A1
(43) Pub. Date: Apr. 18, 2024

(54) PROLYL HYDROXYLASE DOMAIN INHIBITOR TREATMENT TO IMPROVE SURVIVABILITY OF HEMORRHAGIC SHOCK

(71) Applicant: THE GOVERNMENT OF THE UNITED STATES, AS REPRESENTED BY THE SECRETARY OF THE ARMY, Fort Detrick, MD (US)

(72) Inventors: Andrew P. CAP, Hollywood Park, TX (US); James A. BYNUM, La Vernia, TX (US); Xiaowu WU, San Antonio, TX (US)

(21) Appl. No.: 18/379,740

(22) Filed: Oct. 13, 2023

Related U.S. Application Data
(60) Provisional application No. 63/379,518, filed on Oct. 14, 2022.

(51) Int. Cl. A61K 35/19; A61K 9/00; A61K 31/137; A61K 31/437; A61K 31/4418; A61K 31/472; A61K 31/506; A61K 31/513; A61K 31/5377; A61K 35/14; A61K 35/16; A61K 35/18; A61K 38/04; A61K 38/095; A61K 38/36; A61K 38/38; A61K 47/26; A61P 7/04

(52) U.S. Cl. CPC A61K 35/19; A61K 9/0019; A61K 31/137; A61K 31/437; A61K 31/4418; A61K 31/472; A61K 31/506; A61K 31/513; A61K 31/5377; A61K 35/14; A61K 35/16; A61K 35/18; A61K 38/043; A61K 38/095; A61K 38/363; A61K 38/38; A61K 47/26; A61P 7/04

(57) ABSTRACT

A majority of military casualties occur during the pre-hospital period. The cause of death is largely associated with massive traumatic bleeding that leads to organ damage due to sustained hypoxia. Currently, therapeutics are lacking at the point of injury to mitigate hypoxic damage and maintain the survivability of severe hemorrhage prior to reaching medical facilities. This invention addresses that by introducing a method of co-administering prolyl hydroxylase domain inhibitor (PHDi, MK-8617), anti-fibrinolytic agent (tranexamic acid), and bradykinin receptor antagonist (icatibant) as anti-hemorrhage agents in combination with resuscitation fluid treatment with colloid solution of 25% human albumin that has an advantage of relatively small volume requirement to maintain blood volume. In addition, a kit comprising anti-hemorrhage agents that can be easily carried to the battlefield is also provided here. The therapeutic application of those agents on or near the point of injury can stabilize hypoxia inducible factor-1 alpha and enhance blood clot formation, which improves the patient's cellular adaptation to hypoxia and reduce hemorrhage, and thus can decrease organ failure and increase the patient's survivability.

### STATEMENT AS TO RIGHTS OR INVENTIONS MADE UNDER SPONSORED RESEARCH AND DEVELOPMENT

This invention was made with government support from the U.S. Army Institute of Surgical Research, a subordinate organization of the United States Army Medical Research and Materiel Command.

### BACKGROUND

Severe trauma with hemorrhage is a leading cause of death in military casualties. Previous studies in both civilian and military trauma epidemiology suggest that the majority of trauma-induced death occurs during the pre-hospital period (1-3). For example, 87.3% of all injury mortality cases from Operation Iraqi Freedom (OIF) and Operation Enduring Freedom (OEF) combat casualties in 2001 and 2011 occurred prior to arrival at a medical treatment facility (MTF). Among those, 24.3% are due to potentially survivable injury, which is largely associated with hemorrhage (90.9%). Therefore, pre-hospital transport time and treatment capability are both important factors to determine the survivability and outcome of the patient in combat casualty care, and early interventions to impact the outcome of combat casualties with potentially survivable injury should be focused on mitigating bleeding and complications of hemorrhagic shock during pre-hospital care. In addition to early surgical intervention to repair damage and stop bleeding, blood product transfusion to replace blood loss during the pre-hospital period, or even within minutes after injury is crucial to improve the outcome of hemorrhagic shock as it has shown greater association with 24-hour and 30-day survival than delayed transfusion or no transfusion (4). However, blood products including whole blood and blood components are rarely available prior to treatment at a medical facility.

Since Jun. 15, 2009, a new mandate for prehospital transportation in 60 minutes from call to arrival at the facility of treatment has been enforced for US military casualties with critical injuries (5). Although the current short transportation time is aligned with the Golden Hour policy and facilitates medical treatment including resuscitation with blood products within an hour from injury, this only benefits those who survive at 60 min from injury, and will not be possible if air superiority is not achieved. A study retrospectively comparing the outcome before and after the mandate shows that reduction in KIA mortality (killed in action, died before arrival at treatment facility) is not accompanied by a proportional change in DOW mortality (died of wounds, died after arrival at treatment facility), suggesting that rapid evacuation combined with current en-route and facility-based care resulted in survival, but still carries risk for attendant morbidity or DOW mortality status (6) because there is currently no applicable pre-hospital intervention besides limited blood supplies that is able to maintain life by counteracting the pathophysiologic damage caused by trauma/hemorrhagic shock. For future conflicts, the time for receiving surgical procedures and blood product is expected to prolong and exceed the Golden Hour; it is therefore even more important to have certain interventions during the prehospital timeframe prior to evacuation and transportation to a medical treatment facility, which are able to extend survivability and mitigate morbidity during prolonged hemorrhagic shock.

However, there is currently no applicable pre-hospital intervention besides limited resuscitation fluid and blood products. Stabilization of the organ function under hemorrhagic shock will minimize the side effect and improve outcomes of resuscitation with blood products, and surgical interventions. Current “Golden Hour” policy intends to reduce the time between combat injury and receiving definitive care to 60 min or less, and is associated with reduced combat trauma related mortality and morbidity. However, for both military and civilian trauma systems, pre-hospital treatment capability for those with severe hemorrhagic shock at the point of injury needs to be further developed in order to maintain the survivability prior to receiving definitive care including resuscitation of blood products.

### SUMMARY OF THE INVENTION

Currently, there are no known medical devices or therapeutics that can be implemented at the point of injury to extend the survivability of preventable injury related death prior to access to definitive care and blood products.

The current disclosure provides pre-hospital interventions that can be applied at or near the point of injury to reduce mortality and morbidity prior to evacuation and transport to MTF, prior to which the forward surgical team and blood products are available. The therapeutic outcome of a single or bundled intervention(s) consisting of some combination of prolyl hydroxylase domain inhibitor (“PHDi”, e.g., MK-8617), antifibrinolytic agent (e.g., tranexamic acid), bradykinin receptor antagonist (e.g., HOE140 or Icatibant), calcium supplement (e.g., calcium gluconate or calcium chloride), and volume expander/resuscitation fluid (e.g., 25% albumin) with fibrinogen was determined in an established pre-clinical rodent model with trauma/hemorrhagic shock under limited or no blood product, or colloid-based resuscitation, which can be applied to reduce pre-hospital mortality and morbidity in severe hemorrhagic shock.

The effective candidates reduce morbidity and extend survival time by attenuating multiple organ failure, coagulopathy, lactate level, vascular permeability, and restoring cardiovascular dysfunction in severe trauma with hemorrhagic shock. The results presented in this disclosure demonstrate that single or bundled drugs that are easily carried and delivered to the battlefield with minimal requirement for vital sign monitoring under an austere environment, particularly in prolonged care scenarios, are effective at improving outcomes for subjects experiencing hemorrhagic shock.

In particular, the results of the experiments disclosed herein demonstrate: that a single intervention by administration of prolyl hydroxylase domain inhibitors (PHDi), tranexamic acid (TXA), Icatibant, tranexamic acid/Icatibant, or supplement of calcium ion can improve survivability and reduces multiple organ failure in pre-clinical rat models with trauma and hemorrhagic shock; that administration of volume expander 25% albumin alone or bundled with PHDi, TXA, Icatibant, TXA/Icatibant or supplement of calcium can improve survivability and multiple organ failure in pre-clinical rat models with trauma and hemorrhagic shock; and that administration of a bundle of treatments by combinations of 25% albumin, PHDi, TXA, and Icatibant and calcium supplement can additively and synergistically improve the efficacy and outcome as compared to single treatment on survivability and multiple organ failure in a pre-clinical rat models with trauma and hemorrhagic shock.

Based on the foregoing, embodiments of the present disclosure are related tomethods and kits to reduce morbidity and mortality caused by sever hemorrhage or hemorrhagic shock are provided.

In one embodiment, a method is provided that involves the steps of; controlling a hemorrhage in a subject, if possible, and securing airway and breathing; administering resuscitation fluid through intravenous route, optionally administering aliquot of 250 mL repeatedly up to 2000 mL while continuously monitoring a systolic blood pressure, radial pulse, and/or sensorium signs; administering at least one composition comprising a therapeutically effective amount of anti-hemorrhage agent one time or multiple times, and optionally administering vasopressors and/or inotropic agents.

The resuscitation fluid can be whole blood, plasma/red blood cells/platelets (1:1:1), crystalloid solutions such as lactated Ringer's solution; colloid solutions comprising human albumin, hydroxyl ethyl starch (HES), or dextran; hypertonic saline (5 ml/kg NaCl 7.5%) with or without dextran; or oxygen-carrying blood substitutes such as fluorocarbon-based synthetic oxygen carriers and stroma-free cross linked human- or non-human hemoglobin products. The anti-hemorrhage agent is prolyl hydroxylase domain inhibitor (PHDi), antifibrinolytic agent, bradykinin receptor antagonist, or combination thereof that can be co-administered, and the vasopressor or inotropic is vasopressin, norepinephrine, epinephrine, dobutamine, or their synthetic equivalents.

In some particular embodiments, the resuscitation fluid is fresh whole blood or plasma/red blood cells/platelets (1:1:1). In other embodiments, the resuscitation fluid is a colloid solution comprising human albumin, in particular 5-50% human albumin in saline, and in more particular 25% human albumin, and the resuscitation fluid further comprises fibrinogen (45-55 mg/kg).

Further, PHDi is one selected from a group consisting of roxadustat (FG-4592), daprodustat (GSK-1278863), vadadustat (AKB-6548), molidustat (BAY 85-3934), enarodustat (JTZ-951), and MK-8617, and in particular MK-8617, which is administered at a dose of 0.5-5 mg/kg, in particular 1-3 mg/kg. The composition comprising PHDi is typically administered quickly after onset of hemorrhage or administration of resuscitation fluid, typically 5 min to 6 hr after onset of hemorrhage or resuscitation fluid administration. In addition, the composition comprising PHDi is in a solid, semi-solid, or liquid dosage form, and administered orally or intravenously.

The antifibrinolytic agent is one selected from a group consisting of tranexamic acid, aminocaproic acid, and aprotinin, and in particular tranexamic acid, which is administered at a dose of 5-50 mg/kg, in particular 20-30 mg/kg. The composition comprising antifibrinolytic agent is administered is typically administered quickly after onset of hemorrhage, typically 5 min to 6 hr after onset of hemorrhage, or after administering the resuscitation fluid. In addition, the composition comprising antifibrinolytic agent is in a dosage form of solid, semi-solid, or liquid dosage form, and administered through oral, parenteral, or topical route.

The bradykinin receptor antagonist is a bradykinin B2 receptor antagonist, icatibant, which is administered at a dose of 0.1-5.0 mg/kg, in particular 0.4-1.0 mg/kg. The composition comprising bradykinin receptor antagonist is administered is typically administered quickly after onset of hemorrhage, typically 5 min to 6 hr after onset of hemorrhage or after administering the resuscitation fluid. In addition, the composition comprising bradykinin receptor antagonist is in a dosage form of solid, semi-solid, or liquid dosage form, and administered through oral, parenteral, or topical route.

The composition further comprises pharmaceutically suitable excipients.

In addition, the anti-hemorrhage agent or combination thereof are co-administered with calcium supplement, wherein the calcium supplement is calcium gluconate or calcium chloride, which is administered at a dose of 0.01-1 ml/kg, in particular 0.1 ml/kg. The calcium supplement is in a solid- or liquid dosage form for oral or parenteral administration.

Furthermore, a kit for pre-hospital use is provided, which includes; at least one composition comprising a therapeutically effective amount of anti-hemorrhage agent for one time- or multiple time administration, wherein the composition is in a liquid dosage form contained in an injectable container of 5-25 mL volume or solid, semi-solid, or liquid dosage form for oral administration, and optionally comprising non-flammable hand sanitizer, at least one pair of disposable nonlatex gloves, sterile saline for wound washing, and sterilized cleaning pads.

In the kit, the anti-hemorrhage agent is prolyl hydroxylase domain inhibitor (PHDi), antifibrinolytic agent, bradykinin receptor antagonist, or combination thereof that can be co-administered, and wherein the composition may further comprise pharmaceutically suitable excipients in addition to the active ingredient.

The kit further comprises colloid solution of 5-50% human albumin, in particular 25% human albumin, and wherein it may further comprise fibrinogen, and optionally calcium gluconate or calcium chloride.

Although the main objective of the disclosure is for pre-hospital use in the field, the anti-hemorrhage agents can also be used in the surgery room to reduce unnecessary blood loss and promote recovery after surgery.

### BRIEF DESCRIPTION OF THE DRAWINGS

FIG. 1. A graph of mortality rate at battlefield, indicating that 90% preventable injury-related death is associated with hemorrhage.

FIGS. 2A-C. Mitochondria dysfunction (platelets). (FIG. 2A) Routine respiration for 120 min after polytrauma/hemorrhage, (FIG. 2B) Oxidative burst capacity for 120 min after polytrauma/hemorrhage, (FIG. 2C) Intracellular ATP concentration at time points of 0, 0.5, 2, and 4 hrs after polytrauma/hemorrhage. Values represent as means±SEM. *P<0.05 compared with time 0.

FIG. 3. The effect of PHDi prior to polytrauma/hemorrhage on lactate level. PHDi (MK-8617, 1 mg/kg) or vehicle was orally applied in rats prior to trauma (n=3-4/group). The lactate in whole blood samples were collected at baseline (BSL), shed hemorrhage blood (HEM), 2- and 4 hours after trauma and measured by iSTAT. PHDi attenuates lactate levels in rats with polytrauma and hemorrhagic shock. Values represent as means±SEM.

FIG. 4. An illustration of a whole blood resuscitation experimental scheme in a lethal hemorrhagic shock rat model, showing fixed-volume hemorrhage at fast-to-slow pattern.

FIGS. 5A-C. Test results of the lethal hemorrhagic shock in rat models of FIG. 4. (FIG. 5A) Hemorrhage; (FIG. 5B) Survivability; and (FIG. 5C) Lactate level according to the estimated blood volume drained from the rat.

FIGS. 6A-D. The effect of intravenous administration of PHDi after polytrauma/hemorrhage. PHDi (MK-8617, 1 mg/kg, n=8) or vehicle (n=7) was administered intravenously at 20 min after trauma. (FIG. 6A) Mean arterial pressure (MAP); (FIG. 6B) Lactate (hyperlactatemia); (FIG. 6C) Prothrombin time (PT, acute traumatic coagulopathy); and (FIG. 6D) Fibrinogen level.

FIGS. 7A-B. The effect of intravenous administration of PHDi after polytrauma/hemorrhage on hemodynamics (MAP (FIG. 7A), HR (FIG. 7B)).

FIGS. 8A-C. The effect of intravenous administration of PHDi after polytrauma/hemorrhage on hemostasis (PT (FIG. 8A), aPTT (FIG. 8B), and fibrinogen by ST4 (FIG. 8C)).

FIGS. 9A-C. The effect of intravenous administration of PHDi after polytrauma/hemorrhage on metabolism (lactate by iSTAT (FIG. 9A), base excess (FIG. 9C), and HCO3(FIG. 9C)).

FIG. 10. The effect of intravenous administration of PHDi after trauma/hemorrhage on survivability (Kaplan-Meier plots with log-rank test, Fisher's exact test).

FIGS. 11A-C. The effect of anti-fibrinolytic agent (tranexamic acid; TXA) on polytrauma/hemorrhage. Single dose of TXA (10 mg/kg) given prior to trauma. (FIG. 11A) Prothrombin time. Values represent as means±SEM. *: p<0.05, significant difference between TXA and Vehicle; #: p<0.05, significant difference as compared with baseline. (FIG. 11B) Lung wet/dry weight ratio. TXA was given just before trauma (BT, red), just after trauma (AT, green) and 45 min after the end of trauma (yellow). Values represent as means±SEM. *: p<0.05, significant difference as compared with vehicle. (FIG. 11C) Acute lung injury. TXA given 45 min after trauma (and 15 min prior to resuscitation). Levels of platelet and leukocytes (neutrophils and monocytes) infiltration, and C5a and neutrophil elastase in the lung as compared with FWB. *: p<0.05, significant difference between FWB and FWB/TXA; #: p<0.05, significant difference as compared with Non-R; $: p<0.05, significant difference as compared with Non-R, FWB and FWB/TXA. Values represent as means±SEM. Non-R: Non-Resuscitation; FWB: Fresh Whole Blood; TXA: Tranexamic Acid; AT: After Trauma; BT: Before Trauma.

FIG. 12. The effect of bradykinin receptor antagonist (HOE140) on tissue water content/vascular permeability in the brain, lung and skeletal muscle after polytrauma/hemorrhage and resuscitation of lactate Ringer's fluid (20% of Blood Volume). HOE140 or vehicle were given intravenously in rats (n=4/group) at 45 min after trauma (15 min prior to resuscitation of lactate Ringer's fluid). The tissues were harvested 2 hours after trauma. Values represent as means±SD. *: p<0.05 as compared with vehicle.

FIG. 13. Structures of MK-8617, tranexamic acid and icatibant.

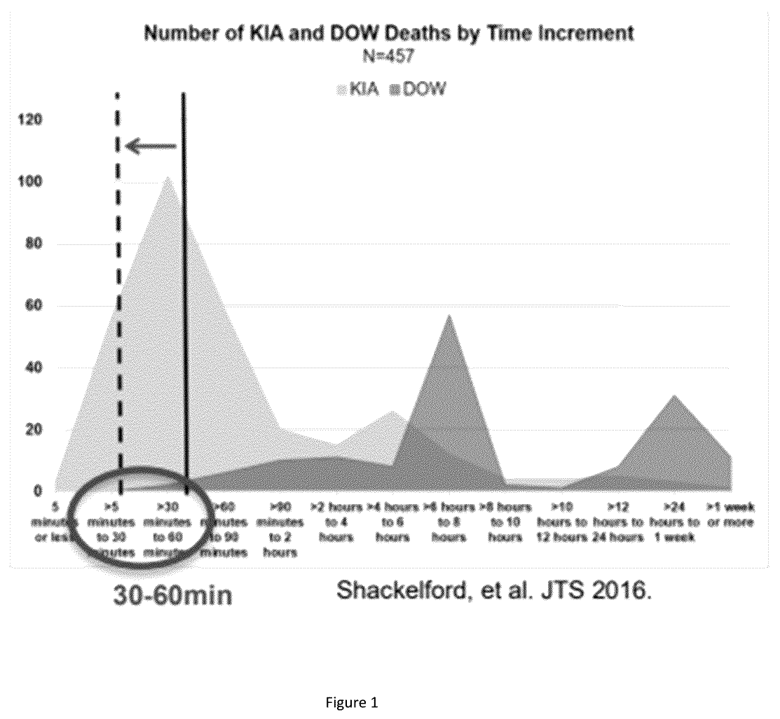

### DETAILED DESCRIPTION

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### A. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about,” means plus or minus 20 percent of the recited value, so that, for example, “about 0.125” means 0.125±0.025, and “about 1.0” means 1.0±0.2.

As used herein, the terms “treatment,” “treating,” and the like, refer to obtaining a desired pharmacologic and/or physiologic effect through administering agent(s) or composition(s). “Treatment,” includes: (a) preventing, partially preventing, reversing, alleviating, reducing the likelihood of, or inhibiting the condition or disease (or symptom thereof) from occurring in a subject. The subject can be those suffering from moderate to severe hemorrhage, or hemorrhagic shock, (b) inhibiting the condition or disease or symptom thereof, such as, arresting its development; and (c) relieving, alleviating or ameliorating the condition or disease or symptom thereof, such as, for example, causing regression of the condition or disease or symptom thereof. Treatment can include administering one or more agents or compositions, performing a procedure such as surgery, blood transfusion and the like, and/or administering necessary drugs or agents. e.g., anti-hemorrhage agents of this disclosure with or without vasopressors or inotropic drugs including but not limited to norepinephrine, epinephrine, dobutamine and vasopressin. Some patients with severe hemorrhage or hypovolemic shock may have respiratory failure and require ventilator assistance including but not limited to biphasic positive airway pressure or intubation and ventilation.

As used herein, the term “subject” or “patient” are used interchangeably to refer to a human or non-human mammal or animal. Non-human mammals include livestock animals, companion animals, laboratory animals, and non-human primates. Non-human subjects also specifically include, without limitation, horses, cows, pigs, goats, sheep, dogs, cats, guinea pigs, hamsters, mink, and rabbits. In some embodiments, a subject is a human patient who has lost at least 30% of blood volume. A “subject in need” is a subject that is at risk of developing sever hemorrhage or hypovolemic shock, or who manifests any characteristics or symptoms of severe hemorrhage or hypovolemic shock.

As used herein, the term “administering” and its cognates refer to introducing an agent to a subject, and can be performed using any of the various methods or delivery systems for administering agents or pharmaceutical compositions, and any route suitable for the composition and the subject, as known to those skilled in the art. Modes of administering include, but are not limited to oral administration, parenteral administration (e.g., intravenous, subcutaneous, intramuscular, intraperitoneal, intrathecal or intradermal injections), or local administration directly into or onto a target tissue. Administration by any route or method that delivers a therapeutically effective amount of the drug or composition to the cells or tissue to which it is targeted is suitable for use with the invention.

As used herein, the term “co-administration,” with respect to administration of more than one composition comprising active agents to a subject refers to administering more than one agent or composition simultaneously or at different times. The one or more active agents can be delivered in one or more pharmaceutical compositions that contain one active agent each, or using pharmaceutical compositions that each contain one or more active agent(s). The different pharmaceutical compositions can be formulated for the same or different routes of administration. The administration of the separate pharmaceutical compositions can be accomplished at the same time, in quick succession, or separated in time by minutes, hours, days, or weeks. A combination pharmaceutical composition contains more than one active ingredient/agent and a pharmaceutically acceptable excipients.

As used herein, the term “effective amount” refers to an amount of an agent or composition that alleviates or reduces cardiovascular, pulmonary, renal, gastrointestinal, or neuronal symptoms in a subject suffering from severe hemorrhage or hypovolemic shock, and decreases organ failure or mortality due to blood loss. As will be appreciated by those of ordinary skill in the art, the absolute amount of a particular agent that is effective may vary depending on such factors as the biological endpoint, the particular active agent, the target tissue, etc. Those of ordinary skill in the art will further understand that an “effective amount” may be administered in a single dose, or may be achieved by administration of multiple doses over a period of time. An effective amount of a pharmaceutical composition that contains an effective amount of one or more agents is an amount of each agent such that the overall composition is effective.

As used herein, the term “shock” or “circulatory shock” refers to a life-threatening condition that occurs when the body is not getting enough blood flow, which causes insufficient supply of oxygen and nutrients to the cells/tissues/organs as well as accumulations of acids and toxic substances so as to damage the cells/tissues/organs. Shock requires immediate treatment and can get worse very rapidly. As many as 1 in 5 people in shock will die from it. The main types of shock include cardiogenic shock (due to heart problems), hypovolemic/hemorrhagic shock (due to diminished blood volume and decreases in the filling pressure of the circulation), anaphylactic shock (caused by histamine mediated allergic reaction), septic shock (due to infections), neurogenic shock (due to loss of vasomotor tone throughout the body and increased vascular capacity). In the case of hemorrhagic/hypovolemic shock, the progress of the shock can be divided into 3 stages: (1) a non-progressive stage in which full recovery is possible by normal compensatory mechanisms (baroreceptor reflexes, central nervous system ischemic response, blood vessel constriction, activation of renin/angiotensin II and antidiuretic hormone for peripheral arteriole constriction and kidney water retention, increase of epinephrine and norepinephrine by adrenal medullae for peripheral arteriole constriction) (2) a progressive stage (cardiac depression, vasomotor failure, increased capillary permeability, release of toxins by ischemic tissues such as histamine, serotonin, and tissue enzymes, accumulation of lactic acid, generalized cellular deterioration, etc.), in which, if not intervened, the shock becomes worse due to a positive feedback, and (3) an irreversible stage in which although the patient is still alive, no known forms of treatment can save the life.

As used herein, the term “excipients” refers to the substances other than the active ingredient or agent, used in pharmaceutical dosage forms. The excipients are considered as inert substances, i.e., they do not have any active role in therapeutics, but they can be used to support the process to produce an effective product. Examples of excipients are active pharmaceutical ingredient excipients, binder excipients, capsule shell excipients, carrier excipients, coating systems excipients, controlled release excipients, diluent excipients, disintegrant excipients, effervescent system excipients, emulsifier excipients, film former excipients, flavor excipients, high-functionality excipients, lipid excipients, lubricant excipients, modified release excipients, penetration enhancer excipients, permeation enhancer excipients, pH modifier excipients, plasticizer excipients, preservative excipients, sachet filling excipients, solubilizer excipients. solvent excipients, surfactant excipients, sustained release excipients, taste masking excipients, thickener excipients, viscosity modifier excipients, blending excipients, filler excipients, compaction excipients, direct compression excipients, dry granulation excipients, hot melt extrusion excipients, wet granulation excipients, rapid release agent excipients, film formation excipients, increased bioavailability excipients, dispersion excipients, solubility enhancement excipients, stabilizer excipients, capsule filling excipients, powder blends excipients, tablet compressibility excipients, etc. (https://www.americanpharmaceuticalreview.com/25335-Pharmaceutical-Raw-Materials-and-APIs/25283-Pharmaceutical-Excipients/)

As used herein, the term “dosage form” refers to pharmaceutical preparations in which a specific mixture of active ingredients of a drug and inactive components (excipients) are formulated in a particular shape or form to facilitated administration and accurate delivery of active ingredients, and/or to be presented in the market. Solid dosage forms include powders, tablets, granules, capsules, cachets, pills, lozenges, suppositories. Semi-solid dosage forms include ointment, creams, paste, gels, poultices. Liquid dosage forms include collodions, droughts, elixirs, emulsions, suspension, enemas, gargles, linctuses, lotion, liniments, mouth washes, nasal drop, paints, solutions, syrups. Gaseous dosage forms include aerosols, inhalations, and sprays. (https://thepharmapedia.com/pharmaceutical-dosage-form-pharmaceutics/pharmacy-notes/)

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin. Aqueous carriers, such as saline solutions, aqueous dextrose and glycerol solutions may be used when the pharmaceutical composition is administered intravenously. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain a minor amount of a wetting or emulsifying agent, or a pH buffering agent.

### B. Overview

Hemorrhage-induced hypovolemic shock is a leading cause of acute death in severe trauma; risk of mortality increases with delays in blood-based resuscitation and with increasing transport times. For hypovolemic shock without or with delayed resuscitation, there is no standardized intervention strategy in clinical practice, which is however urgently required for the readiness of future military forces preparing for combat casualty care in austere environments without established medical theater facilities. This disclosure is a novel pre-hospital anti-shock intervention that can be easily carried and applied at or near point of injury to improve survival during the period of hypovolemic shock from non-compressible life-threatening bleeding, to extend the duration of cellular viability, and to mitigate multiple organ failure prior to evacuation and surgery. The methods and kits of this disclosure represent a potential update to the clinical practice guidelines and enable effective prolonged field care by combat medics.

In this disclosure, inventive methods and a kit are provided for pre-hospital administration of a single- or combination intervention(s), comprising prolyl hydroxylase domain inhibitor (e.g., MK-8617), antifibrinolytic agent (e.g., tranexamic acid), bradykinin receptor antagonist (e.g., HOE140 or Icatibant), calcium supplement (e.g., calcium gluconate or calcium chloride), and volume expander/resuscitation fluid (e.g., 25% albumin) with or without fibrinogen, which can reduce pre-hospital mortality and morbidity in severe hemorrhagic shock.

### C. Hemorrhagic Shock

Hemorrhagic shock is a complex and multifactorial event induced by excessive blood volume loss from the intravascular space. The pathophysiologic changes are determined by the intensity and duration of hypoperfusion, and progressive development from compensation to decompensation which is then irreversible if no treatment is implemented. Hypoperfusion-induced tissue injury can be involved at either cellular, tissue, or whole-organism levels and also interact with each other at different levels.

When hemorrhagic shock is under-compensated, in response to intravascular volume depletion and blood pressure drop, the sympathetic nervous-, neuroendocrine-, and cardiovascular systems play a synergistic role to maintain cardiac output by increasing heart rate, myocardial contraction, and peripheral and splenic vasoconstriction, which diverts blood flow away from the gastrointestinal tract, skin, and skeletal muscle to provide adequate perfusion to the brain and heart (7, 8). In the kidney, renal perfusion can be maintained under low to moderate hemorrhage, but declines with severe hemorrhage due to decreased blood flow and activation of the renin-angiotensin system (8).

In decompensated shock, the cardiac output and blood pressure both drop due to tissue hypoxia, and acidosis overwhelming sympathetic stimulation; the shock quickly becomes irreversible when cardiac output and blood pressure cannot be restored by resuscitation with volume replacement. At the tissue level, cellular damage is caused by the decline in oxygen delivery from hypoperfusion, especially in cells of the liver, gut and kidney where hypoperfusion occurs earlier than in the brain and heart due to compensative mechanisms. If the shock stage continues without intervention, increased anaerobic respiration and cellular dysfunction will lead to buildup of lactate, free radicals, and insufficient adenosine triphosphate (ATP) production, which results in enhanced acidosis, oxidative stress, and ultimately cell death and organ failure. When tissue perfusion is re-established, the burst generation of free radicals from reactivation of cellular respiration and oxygen consumption can cause tissue damage (ischemia/reperfusion injury). Additionally, accumulated cellular degradation products (damage associated molecular patterns, DAMPs) and inflammatory mediators enter the systemic circulation, thereby amplifying the inflammatory response to shock and injury, which contribute to late mortality after hemorrhagic shock (9).

The mechanism of hemorrhagic shock-induced pathologic changes has been broadly studied. Fundamentally, under the low oxygen environment caused by shock-ischemia, physiologic aerobic metabolism is persistently shifted to anaerobic metabolic adaptation, which alters internal homeostasis and ultimately contributes to the pathologic responses of inflammation, coagulation, mitochondrial dysfunction, cellular apoptosis and organ failure.

Liquid chromatography-mass spectrometry (LC-MS)-based metabolomics analysis has been used to identify the metabolic derangements in response to trauma/hemorrhagic shock (10). In particular, hemorrhagic shock leads to the accumulation of mitochondrial tricarboxylic acid (TCA) cycle intermediates, such as fumarate and succinate, which are capable of mediating various pathophysiologic changes including metabolic acidosis, inflammation, and coagulopathy (11). Most importantly, the accumulation of succinate generates mitochondrial reactive oxygen species (ROS) via reverse electron transport through mitochondrial complex I upon reperfusion (12), and initiates ischemia/reperfusion injury. It is recently reported that administration of cell-permeable succinate prodrugs can bypass mitochondrial complex I and improve mitochondria dysfunction due to mitochondrial complex I deficiency in pediatric mitochondrial disease (12-1).

### D. Hypoxia-Inducible Factor-1 Alpha (HIF-1α)

The accumulation of TCA intermediates is also linked to the hypoxic response by upregulating hypoxia-inducible factor-1 alpha (HIF-1α) (13), which includes stabilizing HIF through inhibition of prolyl hydroxylase domains (PHDs), succinate dehydrogenase (SDH) and fumarate hydratase (FH) (14, 15).

HIF-targeted genes mediate multiple biological functions including angiogenesis, apoptosis, coagulation, DNA damage and repair, cell proliferation, and metabolism, which may ultimately regulate systemic tolerance and adaptation to hypoxia, and determine the fate of cell survival or death under prolonged hypovolemic conditions (16, 17).

### E. Effective Anti-Shock Candidates

### E.1. Targeting Pathologic Change in Response to Hypoxia-Prolyl Hydroxylase Domain Inhibitors (PHDi)

Prolonged tissue hypoxia and reduction of oxygen utilization by cellular mitochondria leads to accumulation of lactate in the tissues as a result of anaerobic glycolysis. Elevation of lactate and reduction of lactate clearance is an independent predictor of death in various groups of critically ill patients, including patients with trauma (18, 19). Clinically, a serum lactate greater than 2.5 mmol/L in 12 hours after injury predicts development of multiple organ failure (MOF) (20). Under hypoxic conditions, hypoxia inducible factor (HIF) is essential to facilitating hypoxia tolerance and adaptation through activating a transcriptional cascade to regulate cellular metabolism and maintain tissue homeostasis (21, 22).

HIFs are alpha-beta heterodimeric transcription factors, which are rapidly degraded under normal oxygen levels by oxygen-sensing prolyl hydroxylase domains (PHDs) and binding of the von Hippel-Lindau (VHL) gene product (23, 24). PHDs require utilization of oxygen as a substrate for ubiquitination and proteasomal degradation of HIFs so that the activity of PHDs is inhibited under hypoxia which in turn stabilizes HIFs (25, 26).

The HIF-1-alpha-beta complex translocates into the nucleus and binds to hypoxia-responsive elements (HRE) of target genes (27, 28) whose products play an adaptive role in response to hypoxic conditions. Consequently, HIF-1 promotes cellular metabolic reprogramming to establish the adaptation to hypoxia and resistance to cell death, which is involved in modulating gene expression of enzymes including those involved in the glycolysis pathway, oxygen utilization of mitochondria, and clearance of mitochondria ROS (29-34). HIF-1 not only enhances glycolysis and lactate efflux from the cells, but also increases uptake of lactate for gluconeogenesis (Cori cycle), resulting in increased lactate clearance and diminished lactic acidosis under hypoxia (35).

Therefore, PHD inhibitors, which stabilize HIF, are being developed for renal failure, tissue ischemia, and tissue regeneration. Administration of PHD inhibitor (PHDi) significantly improves survival for metformin-associated lactic acidosis (36, 37), prevents mitochondrial injury in the ischemia reperfusion model, and reduces lactate in LPS induced septic shock (35). Additionally, stabilization of HIF by PHDi is beneficial to reducing acute lung injury and acute kidney injury by maintaining endothelial cell integrity and hypoxia tolerance under oxygen stress during septic shock or ischemia reperfusion (39, 40). Currently, oral application of three PHDi (HIF activators) is promoted in the clinical trials of acute kidney injury: vadadustat (AKB-6548), roxadustat (FG-4592) and daprodustat (GSK-1278863). However, the application of PHDi in the treatment of hemorrhagic shock-induced hypoxia has not been investigated.

Examples of this disclosure demonstrates a novel therapeutic application of PHDi as a single, or complimentary, anti-shock therapeutics prior to resuscitation with blood products or other fluids in severe trauma and hemorrhagic shock. Hypoxia inducible factor (HIF) is a master regulator for cellular adaptation to low oxygen environments. Oxygen availability affects oxygen-dependent activity of prolyl hydroxylation of HIF-1α to determine whether HIF-1α is subsequently degraded in proteasome under normoxia, or is protected from degradation and translocated into the nuclei of cells to initiate gene expression and post-translational modification under hypoxia.

Severe hemorrhagic shock leads to tissue hypoperfusion that eventually damages organ function due to hypoxia. Inhibition of prolyl hydroxylase activity prevents HIF-1α degradation, which improves cellular adaptation to hypoxia and ultimately conducts a series of signals for survival. Additionally, prolyl hydroxylase domain inhibitor (PHDi) plays a role for metabolic reprogramming to enhance the uptake of lactate to ameliorate lactic acidosis, a frequent complication of hemorrhagic shock, severe infectious and ischemic diseases.

Currently, various formulations of PHDi are under clinical trials to treat anemia of chronic kidney disease by increasing endogenous production of erythropoietin. Inhibition of prolyl hydroxylase activity protects HIF-1I from degradation, which improves reticulocyte production and maturation by promoting erythropoietin (EPO) production in the kidney and improving iron absorption by inhibition of hepcidin production in the liver. PHDi will be a useful surrogate to treat hemorrhagic anemia when blood products are constrained.

The disclosed invention was conducted in rodent models of hemorrhage or polytrauma/hemorrhage. The compound MK-8617 was initially tested in this invention. MK-8617 was purchased from Selleckchem.com. MK-8617 was dissolved in DMSO/PEG400 with normal saline and stored in −20-0° C.

This is the first time PHDi was tested as an anti-shock agent for treatment of severe trauma and hemorrhagic shock. Current therapeutic strategy is based on the physiologic mechanism of cellular adaptation to low oxygen environments and the fundamental cause of death due to organ dysfunction that is induced by hypoperfusion during hemorrhagic shock and not corrected by fluid or blood resuscitation. The outcome of acute administration of PHDi was tested in two different rodent models that are translatable to various clinical scenarios of trauma and hemorrhagic shock. The results suggest that, for those died within one hour due to hemorrhagic shock, a single dose of PHDi is capable of extending survival time and improving outcomes followed by whole blood resuscitation; and, for those with severe trauma and hemorrhage without access to fluid resuscitation due to prolonged evacuation or transportation time, a single dose of PHDi may reduce the rise of lactate and acute traumatic coagulopathy.

### E.2. Targeting Hyperfibrinolysis and Vascular Permeability-Tranexamic Acid (TXA) and Icatibant

Hemorrhagic shock is enhanced under severe trauma with life-threatening active bleeding, which is more common with acute traumatic coagulopathy (ATC). The cause of ATC is multifactorial and attributed by massive consumption of coagulation factors, excess elevation of fibrinolytic activity (elevation in plasma d-dimers (41) and tissue plasminogen activator (tPA)) (42), endothelial damage, platelet dysfunction, and hemodilution. Hypothermia, acidosis and coagulopathy comprises the bloody vicious cycle used to describe the deadly stage in hemorrhagic shock. Trauma associated with coagulopathy results in an increase in transfusions, organ dysfunction, and critical care unit days, and occurs in 25% and 38% of severely injured civilian and military trauma populations, respectively (43, 44).

Tranexamic acid (TXA) is a lysine derivative that exerts antifibrinolytic capability by blocking the lysine binding site of plasminogen or plasmin (45). TXA reduces activation of plasmin from plasminogen by preventing plasminogen from associating with tPA on the surface of fibrin strands. Administration of TXA has been demonstrated to reduce blood loss in patients undergoing major surgery and postpartum hemorrhage (46, 47), suggesting that the principal mechanism of TXA is associated with reduction of fibrinolysis and improvement of clot stability at the surgical or injured site. Additionally, recent clinical studies have shown that TXA improves survival rates in trauma patients (CRASH-2) (48, 49). This beneficial effect has also been demonstrated in studies among military casualties (50, 51). Even though TXA is currently used off-label in trauma patients, it has been strongly recommended for use in this population (52). Clinical data (prophylactic use in elective surgery and CRASH-2 in trauma) also suggest that timing of administration in relation to coagulation and immune system activation caused by surgery or trauma may determine biological effects.

TXA has also been shown to have pharmacokinetic bioequivalence when using different routes of delivery (IM, IV, IO, PO, topical) (53-57), and has been documented to be safe for use in the pre-hospital setting for trauma (58, 59). Administration of TXA is recommended for trauma as described in current Tactical Combat Casualty (TCCC: TXA is given no later than 3 hr. 1 gm IV (100 ml) for 10 min, begin the second infusion of 1 gm TXA after initial fluid resuscitation has been completed.) guideline. However, the interaction of TXA with other anti-shock therapy has not been investigated previously.

Plasmin is known not only to affect fibrinolysis in fibrin clots but also to modulate inflammation and vascular integrity in tissues. It has been demonstrated previously that there is an increase in plasminogen receptor expression on the surface of activated monocytes and platelets after trauma and sepsis. Circulating plasminogen binds to plasminogen receptors through lysine binding sites, being converted to plasmin by increased activity of tPA (61-63). Plasmin further causes tissue inflammation by promoting cellular transmembrane migration, extracellular matrix degradation (63), and damages vascular integrity and increases vascular permeability through activation of bradykinin signaling (64).

TXA attenuates tissue inflammation and organ dysfunction possibly not by changing circulatory plasmin activity (circulatory plasmin activity was not significantly changed by TXA administration in current study), but rather by reducing plasmin formation on activated monocytes and platelets by blocking circulatory plasminogen binding to plasminogen receptors on those cells. Additionally, TXA may also directly protect endothelial integrity and prevent or ameliorate endotheliopathy occurring from trauma and hemorrhagic shock (65, 66).

Activation of plasmin can generate bradykinin by activating factor XII, which in turn activates prekallikrein to kallikrin, and then cleaves high molecular weight kininogen to release bradykinin. Bradykinin produces vasodilation and increased vascular permeability, fever, and hypotension through stimulation of endothelial nitric oxide and prostaglandin formation, in addition to effects on regulation of neutrophil trafficking and nociception (64, 67). In particular, bradykinin causes vasodilation and increased vascular permeability through the bradykinin receptor on the endothelium. Recent studies demonstrate that blockade of bradykinin effects by using a bradykinin receptor antagonist attenuates elevation of blood-brain barrier permeability induced by tPA (64, 68), which fills the gap of our understanding of how the fibrinolytic system, through bradykinin, drives changes in vascular structure and end-organ function. It has been well documented in both human and animal studies that shedding of endothelial glycocalyx in response to shock and systemic inflammatory is the fundamental pathophysiologic alteration of endotheliopathy (69), and elevation of circulatory syndecan-1 is identified in severe trauma populations and has been used as an important biomarker for diagnosis of endotheliopathy (70).

The plasmin-bradykinin axis further improves our understanding of the mechanism of hemovascular dysfunction in response to trauma and hemorrhagic shock. It is theorized that application of plasmin or bradykinin blockade, or a combination, can provide a valuable addition to current inadequate management in trauma induced endotheliopathy and remote organ failure.

Although the improvement in trauma mortality by TXA has been demonstrated in the CRASH-2 trial, the mechanism is still unclear due to lack of additional measurements. Our previous studies demonstrate that rats subjected to severe trauma also develop an elevation over time in tPA, plasmin activity and d-dimers (71) with a subsequent fall in clot strength (72), and inhibition of plasmin activity by administration of the anti-fibrinolytic agent tranexamic acid attenuates elevation of water content (vascular permeability) in the lung after resuscitation in rats with polytrauma and hemorrhagic shock (73). Early resuscitation with plasma can attenuate vascular permeability via maintenance of glycocalyx integrity after shock (74).

Endotheliopathy is associated with vascular permeability, coagulation, and the inflammatory cascade, which certainly is an important target for anti-shock treatment. Combination therapy with TXA (anti-plasmin), Icatibant (bradykinin blocker), and FDP or 25% albumin (protect glycocalyx integrity) may have significant impact on restoration of hemovascular function.

### E.3. Targeting Hypocalcemia Associated Cardiovascular Dysfunction and Coagulopathy-Calcium Gluconate or Chloride

The serum calcium concentration consists of three forms; ionized, complexed and protein-bound (with albumin). Only free ionized calcium participates in cellular function. Calcium has crucial, wide-ranging effects on coagulation, membrane potentials and neuronal excitability, skeletal/cardiac/smooth muscle contractility, and the release and activity of various hormones and neurotransmitters. Hypocalcemia commonly follows hypovolemic shock (75, 76), especially in patients receiving massive transfusion (77-79) due to additional chelation of serum Ca²⁺ by the citrate from blood products. Citrate is normally metabolized in the liver, which is affected if liver function is impaired by prolonged hypoperfusion during shock.

Calcium is an important cofactor in coagulation factors (factors II, VII, IX and X along with protein C and S) in the coagulation cascade, and also serves as an important signal transmitter for platelet functions (adherence, aggregation and secretion). Hypocalcemia along with acidosis and hypothermia contributes to development of acute traumatic coagulopathy (80), and hypocalcemia is associated with increased bleeding potential after trauma (81, 82), which can worsen with massive transfusion (83).

Calcium plays an important role in neuromuscular and cardiovascular membrane stability, cardiac conduction, myocardial contractility, and vascular reactivity. Severe hemorrhagic shock leads to cardiac dysfunction and vascular hyporeactivity, manifested by reduced cardiac contractility and vascular reactivity to resuscitation, vasoconstriction or vasodilators (84), which may consequently affect capability of compensation and responsiveness to fluid resuscitation and thus intensify tissue ischemic/hypoxic damage or potential over-resuscitation. It is reported that there is dramatic improvement in blood pressure, pulse pressure, and peripheral perfusion when seriously injured hypocalcemic soldiers received supplemental calcium gluconate (or calcium chloride), suggesting that supplementation of Ca²⁺ improves cardiac contractility and vascular responsiveness to hemorrhagic shock (85).

There are a number of studies that demonstrate the association between hypocalcemia and poor outcome in different circumstances among medical, surgical and trauma patients (86). However, recent studies suggest that only severe hypocalcemia (<0.8-09 mmol/L) is independently associated with mortality (87-89), which is consistent with the study suggesting that calcium desensitization is associated with vascular hypo-reactivity after hemorrhagic shock (90). On the other hand, some studies also demonstrate that calcium may compete with magnesium and serve as an inhibitor of membrane Na—K dependent ATPase as well as the mitochondrial ATPase (91), which may interfere with cellular functionality. The hypocalcemia after hemorrhagic shock is in part due to increased Ca²⁺ influx to cells (92, 93), especially under ischemia reperfusion injury (94).

Elevation of intracellular levels of Ca²⁺ and mitochondrial Ca²⁺ uptake may compromise mitochondrial electron transport activities and trigger membrane permeability changes that allow for release of cytochrome-C and other mitochondrial apoptotic proteins into the cytosol to induce cell apoptosis (95). Therefore, there has been long-standing debate as to whether calcium administration is fundamentally beneficial during the acute stage of hemorrhagic shock. Currently, guidelines for Ca²⁺ supplementation in the management of trauma and hemorrhagic shock, and the dose, timing and safety of Ca²⁺ administration, especially during the pre-hospital stage, require further clarification. Clinically, supplementation of Ca²⁺ is based on maintaining serum ionized calcium concentration at least less than 0.9 mmol/L, as a threshold of Ca²⁺ at less than 0.6-0.7 mmol/L is considered adequate for clot formation (80). However, because the Ca²⁺ level is not readily monitored during the pre-hospital stage, a safe range of dosing needs to be determined in order for Ca²⁺ to serve as an important supplement for maintaining cardiovascular functionality and coagulation capability during severe trauma/hemorrhagic shock.

### E.4. Targeting Hypovolemia-25% Albumin:

The inadequacy of fluid resuscitation and relative unavailability of blood products is one of the major obstacles to treatment of shock for military casualties during the pre-hospital stage, and the future scenario of prolonged field care. According to TCCC guidelines, fresh whole blood (FWB) is the first choice for trauma resuscitation, and component therapy in a 1:1:1 ratio of packed red blood cells, plasma, and platelets is recommended when FWB is unavailable. In circumstances where blood components are unavailable, the guideline recommends using volume expanders in the sequence of fresh plasma or FDP (hemostatic and oncotic), synthetic colloid hydroxyethyl starch (HEXTEND® (non-hemostatic but oncotic)) and balanced crystalloid fluid (neither hemostatic nor oncotic) as a last choice.

Pre-hospital administration of FDP or other plasma is beneficial for injured patients at risk for hemorrhagic shock as shown especially in reduced 30-day mortality (96). However, FDP is currently only available to certain SOF units, and it is unknown if it will become widely available soon (97). For a combat medic of conventional forces on the battlefield today, HEXTEND® and crystalloid solutions are the only options for fluid resuscitation. Crystalloid solution only leads to transient volume expansion due to increased vascular permeability under hemorrhagic shock (98). Crystalloid solution leads to serious morbidities caused by hemodilution, coagulopathy and extravascular edema. With prolonged evacuation times, colloid solutions such as HEXTEND® use less total fluid weight and volume than crystalloid solutions to achieve the same amount of intravascular expansion (99), and are beneficial in maintaining volume status with longer intravascular dwell time as compared with crystalloid solutions. However, HEXTEND® is associated with acute kidney injury, anticoagulant effect, and increased mortality in trauma (100-102).

As a substitute to FDP, albumin is an FDA approved and commercially available volume expander that had extensively been used in World War II and Korea where standard blood products were unavailable. In comparison with other blood products, transfusion of albumin does not require advanced preparation, time, resources, and personnel for donor identification, collection, and infusion; all of which may be difficult under operational circumstances (103). Thus, albumin as a volume expander at low-volume is considered the most efficient approach to save the lives of those with prolonged hemorrhagic shock.

However, use of colloid solutions for days to weeks in the ICU setting is clearly different from limited use in the pre-hospital environment, and thus these results are difficult to extrapolate to the field. Albumin use in the ICU setting has long been controversial (104). Recent studies in an animal model with traumatic hemorrhagic shock suggest that 25% albumin administration in a rabbit with trauma and hemorrhagic shock demonstrates a similar survival benefit as plasma, but improves mortality with less volume requirement (⅕ of the volume with iso-oncotic colloid fluid) (105, 106). A systematic review of hyperoncotic albumin usage in a variety of patients found some evidence for a reduction in morbidity, renal impairment, and edema with the use of 20%-25% albumin (107). More importantly, it did not identify any safety concerns or increased morbidity in patients who received hyperoncotic albumin. In the setting of limited use for pre-hospital resuscitation of combat wounded, it is likely that albumin is at least as effective as HEXTEND®, without the danger of increased kidney injury. Also, albumin is already present in the U.S. Department of Defense supply system.

Albumin also offers some logistical advantages over FDP. It does not require reconstitution and is available in a compact, ready to use format, does not require a specialized filter, and standard plastic bag is available. Hyperoncotic 25% albumin provides much of its desired effect by drawing in extravascular fluid to the intravascular compartment. Albumin also offers a potential advantage over FDP in terms of infection risk. Hyperoncotic albumin has a nephroprotective effect, whereas an association is found between HEXTEND® and nephrotoxicity.

In regards to concerns for the safety of albumin in TBI, albumin is the most abundant protein in plasma, which does not cause any side effects in TBI itself. 25% albumin, augmented with fibrinogen concentrate and tranexamic acid to mitigate hemodilution effects on coagulation capacity, offers an effective volume resuscitation alternative that could save lives on the battlefield immediately (108). In this disclosure, co-administration of a combination of 25% albumin along with other pre-hospital anti-shock drugs to mitigate morbidity and improve survivability of severe trauma with hemorrhagic shock is suggested.

### F. Experimental Design

### F.1. Evaluation of Efficacy of Single Intervention

The first specific aim of the experiments disclosed here is to determine whether a single intervention by administration of prolyl hydroxylase domain inhibitors (PHDi), tranexamic acid (TXA), Icatibant, tranexamic acid/Icatibant, or supplement of calcium ion can improve survivability and reduces multiple organ failure in pre-clinical rat models with trauma and hemorrhagic shock.

### (1) PHDi

Hypoxia inducible factor-1alpha is a master regulator to facilitate cellular adaptations to low oxygen environment. The stability of HIF is controlled by the activity of prolyl hydroxylase domain (PHD) protein. Early administration of PHDi in trauma/hemorrhagic shock is expected to improve tissue tolerance to hypoxic damage by stabilization of HIF through inhibition of HIF clearance. Most importantly, HIF-1alpha not only enhances glycolysis and lactate efflux from the cells, but also increases the uptake of lactate for gluconeogenesis (Cori cycle), resulting in acceleration of lactate clearance and amelioration of lactate acidosis under hypoxia. Currently, PHDi is under different phases of clinical trials for treatment of anemia due to chronic kidney diseases as PHDi increases endogenous production of eryt hoursopoietin, which stimulates production of hemoglobin and red blood cells. This may also provide an additional long-term benefit for those who developed chronic anemia due to kidney failure from ischemic injury due to prolonged hemorrhagic shock.

Since PHDi has been developed for clinical trials, the dose and toxicity have been determined in various species including rodent. The PHDi is given immediately after hemorrhagic shock in both acute lethal and acute sub-lethal models of polytrauma/hemorrhagic shock. Although oral route is recommended for PHDi in the treatment of chronic kidney diseases, an intravenous route of application is also proposed if the oral route cannot achieve maximum effect when given after shock. A new compound of PHDi (MK-8617) that is available for both oral and intravenous application at a dose of 0.5 mg/kg (128) (diluted into 500 microliter with diluent (5% DMSO)) was tested. The same volume of diluent was applied as a vehicle used in the control group.

### (2) TXA w/o Icatibant:

Attenuation of vascular permeability can stabilize intravascular blood volume and pressure, and reduce extravascular edema, which thus restores tissue perfusion and attenuates tissue ischemia during hemorrhagic shock. One of the controllers of vascular permeability is bradykinin that is regulated by plasmin. The connection between (bradykinin) and hyperfibrinolysis (plasmin) to regulate vascular permeability was recently demonstrated.

In this disclosure, Icatibant (bradykinin receptor antagonist), tranexamic acid (TXA, anti-fibrinolytics) or the combination of Icatibant and TXA (diluted into 500 microliter with normal saline respectively, and the similar volume of normal saline as a vehicle in the control group) were given immediately after hemorrhagic shock respectively. The doses of TXA (1 g/bolus, IV) and Icatibant (3 mg/bolus, SC) have already been determined in humans clinically, which is estimated 10 mg/kg of TXA, and 0.4 mg/kg of Icatibant as an equivalent dose in rats respectively.

### (3) Supplement of Calcium Ion (Calcium Gluconate/or Calcium Cloride):

Hypocalcemia is common after trauma and hemorrhagic shock, especially in those with massive transfusion of blood products. Supplementation of calcium ion is expected to improve cardiovascular responsiveness in prolonged hypovolemic shock. The calcium ion is commonly supplied as 10% calcium gluconate and infused by a bolus clinically. The calcium gluconate (0.1 ml/kg, diluted into 500 microliter with saline, and the similar volume of normal saline as a vehicle in control group) was given immediately after trauma/hemorrhagic shock, and tested in both acute lethal and sub-lethal model. If calcium gluconate is unavailable (there have been interruptions in supply causing clinically significant shortages) a corresponding dose of calcium chloride can be used.

### (4) Rat Assignment

For the first specific aim of the experiments described above, the experimental sub-groups were assigned as follows in both acute lethal and sub-lethal models respectively. For the surviving animals at 60 min after hemorrhagic shock or trauma, they received none or resuscitation of FWB (20% of estimated blood volume, equivalent to 2 units of whole blood in human). The blood samples were collected at baseline, 60 min, and 4 hours prior to euthanasia, and the tissues (brain, heart, liver, kidney, lung, and small intestines) were harvested immediately from euthanized rats. The multiple organ failure and other measurements including biochemical, apoptosis, mitochondria functionality, hypoxic pathway and metabolites (Krebs cycle and glycolysis), were measured.
Group-I: Vehicle control (DMSO) for PHDi
Group-II: Vehicle control (saline) for TXA, Icatibant, and calcium gluconate
Group-III: PHDi
Group-IV: TXA
Group-V: Icatibant
Group-VI: TXA+Icatibant
Group-VII: Calcium gluconate
Group-VIII: Vehicle control (DMSO)+FWB (at 60 min)
Group-IX: Vehicle control (Saline)+FWB (at 60 min)
Group-X: PHDi+FWB (at 60 min)
Group-XI: TXA+FWB (at 60 min)
Group-XII: Icatibant+FWB (at 60 min)
Group-XIII TXA+Icatibant+FWB (at 60 min)
Group-XIV: Calcium gluconate+FWB (at 60 min)

### F.2. Evaluation of Administration of Volume Expander

The second specific aim of the experiments disclosed here is to determine whether administration of volume expander 25% albumin or bundled with PHDi, TXA, Icatibant, TXA/Icatibant or supplement of calcium can improve survivability and multiple organ failure in pre-clinical rat models with trauma and hemorrhagic shock. In severe hypovolemic shock, administration of a volume expander as early as possible is crucial for survival. Fresh dried plasma (FDP) is an iso-oncotic colloid fluid with necessary fibrinogen and coagulation factors, which has been demonstrated to improve survivability and outcomes in trauma and hemorrhagic shock (96). 25% albumin is a hyper-oncotic colloid fluid and is a currently commercially available resuscitation fluid for hemorrhagic shock. Until FDP becomes available, 25% albumin can be used as a substitute for FDP. On the other hand, 25% albumin is ready for use without the need for reconstitution and a transfusion filter as with FDP. Most importantly, since the oncotic pressure is four times higher than in FDP, only one fourth of the volume is needed. However, for treatment of acute traumatic coagulopathy, concentrated fibrinogen or cryoprecipitate is considered as an important supplement to be used with 25% albumin to avoid dilutional hypofibrinoginemia. Currently, HEXTEND®, as an oncotic resuscitation fluid, is still carried in combat medics so that outcome comparison of 25% albumin to HEXTEND® and plasma was tested in both animal models. 25% albumin (5% of blood volume), HEXTEND®(20% of blood volume), or plasma (20% of blood volume) were administrated intravenously as a bolus immediately hemorrhagic shock. Additionally, the effective single intervention(s) in terms of improvement of survivability or attenuation of multiple organ failure or both were selected and bundled with 25% albumin administration. The potential synergistic effect was determined as compared with 25% albumin administration alone.

The experimental sub-groups were assigned as follows in both acute lethal and sub-lethal models respectively. For the surviving animals at 60 min after hemorrhagic shock or trauma, they either received none or resuscitation of FWB (20% of estimated blood volume, equivalent to 2 units of whole blood in human). The blood samples were collected at baseline, 60 min, and 4 hours prior to euthanasia, and the tissues (brain, heart, liver, kidney, lung, and small intestines) were harvested immediately from euthanized rats. The multiple organ failure and other measurements including biochemical, apoptosis, mitochondria functionality, hypoxic pathway and metabolites (Krebs cycle and glycolysis), were measured.
Group-II: Plasma
Group-IV: HEXTEND®
Group-VI: 25% albumin
Group-V: 25% albumin/fibrinogen concentrate
Group-III: Plasma+FWB (at 60 min)
Group-V: HEXTENT®+FWB (at 60 min)
Group-VII: 25% albumin+FWB (at 60 min)
Group-VIII: 25% albumin/fibrinogen concentrate+FWB (at 60 min)
Group-X: 25% albumin bundled with effective drug selected from the specific aim 1
Group-XI: 25% albumin bundled with effective drug selected from the specific aim 1+FWB (at 60 min)

### F3. Evaluation of Additive or Synergistic Effects of Bundled Treatments

The third specific aim of the experiments disclosed here is to determine whether administration of a bundle of treatments by combinations of 25% albumin, PHDi, TXA, and Icatibant and calcium supplement can additively and synergistically improve the efficacy and outcome as compared with single treatment on survivability and multiple organ failure in a pre-clinical rat models with trauma and hemorrhagic shock.

It is proposed here that a bundle of treatments for treating severe trauma with hemorrhagic shock during the pre-hospital stage of care especially when blood products are unavailable. The therapeutic strategies are focused on targeting the basic pathophysiologic changes in response to hypovolemic shock. With administration of limited volume of volume expander with 25% albumin, the combination of PHDi, TXA, Icatibant, and calcium supplement is expected to extend the survival time by improving systemic tolerance to hypoxia, reducing metabolic lactate, attenuating vascular permeability, restoring hemostasis, and improving cardiovascular responsiveness and functionality. The treatment bundle are applied intravenously as a bolus immediately after hemorrhage shock, and the mortality and outcomes are compared with single intervention as described in Specific Aim 1. The combination of 25% albumin with a single drug has been proposed at Specific Aim-2. Any drug that shows no effect on either improvement of mortality or attenuation of multiple organ failure as shown from specific aim-1 and 2 are not included in the bundle treatment. The outcome of bundle treatment are also measured in survivals with resuscitation of FWB at 60 min after hemorrhagic shock.

The experimental sub-groups are assigned as follows in both acute lethal and sub-lethal models respectively. For the surviving animals at 60 min from hemorrhagic shock or trauma, they either receive none or resuscitation of FWB (20% of estimated blood volume, equivalent to 2 units of whole blood in human). The blood samples are collected at baseline, 60 min, and 4 hours prior to euthanasia, and the tissues (brain, heart, liver, kidney, lung, and small intestines) are harvested immediately from euthanized rats. The multiple organ failure and other measurements including biochemical, apoptosis, mitochondria functionality, hypoxic pathway and metabolites (Krebs cycle and glycolysis), are measured.
Group-I: Vehicle control (include DMSO)
Group-II: Vehicle control (not include DMSO)
Group-II: Bundled intervention (potential candidates: PHDi+TXA+Icatibant+calcium gluconate)
Group-III: Bundled intervention (potential candidates PHDi+TXA+Icatibant+calcium gluconate)+25% albumin
Group-III: Vehicle control (include DMSO)+FWB (at 60 min)
Group-IV: Vehicle control (not include DMSO)+FWB (at 60 min)
Group-IV: Bundled intervention (potential candidates: PHDi+TXA+Icatibant+calcium gluconate)+FWB (at 60 min)
Group-V: Bundled intervention (potential candidates: PHDi+TXA+Icatibant+calcium gluconate)+25% albumin+FWB (at 60 min)

### G. Summary of Results

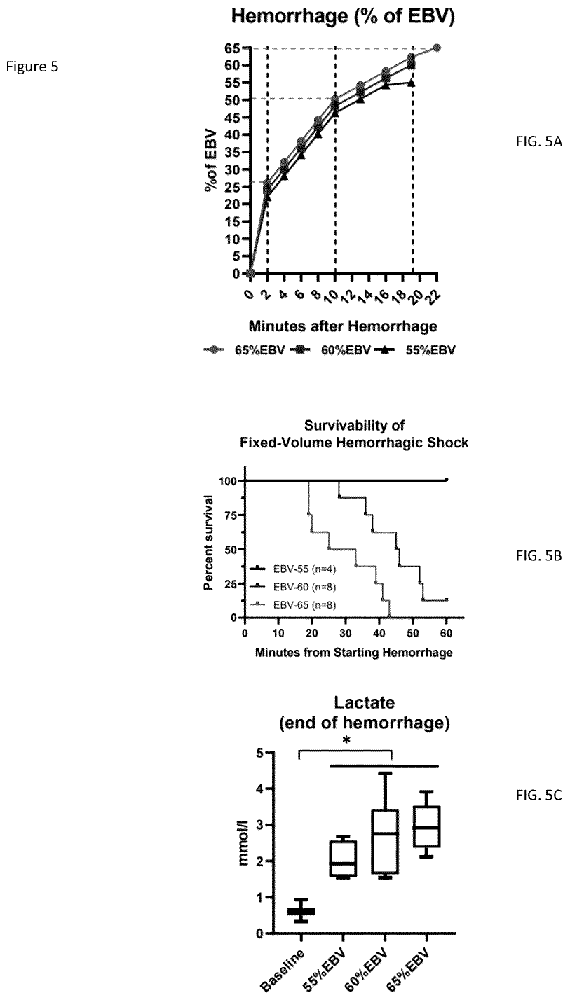

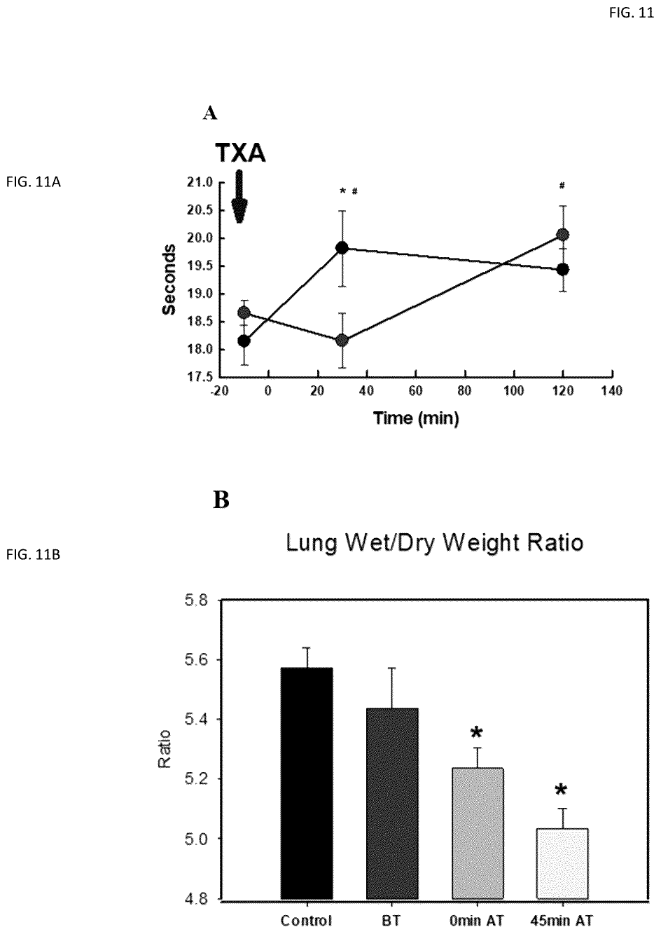

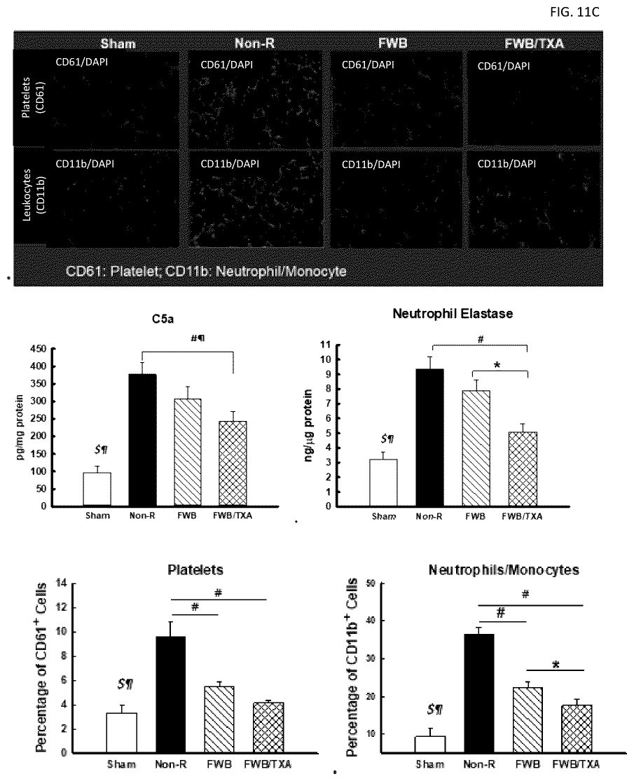

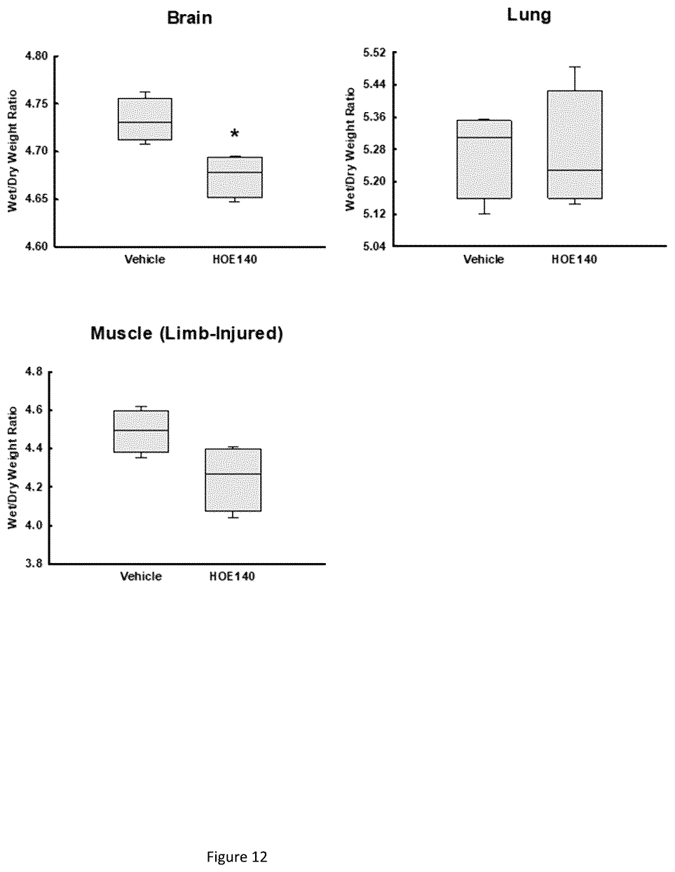

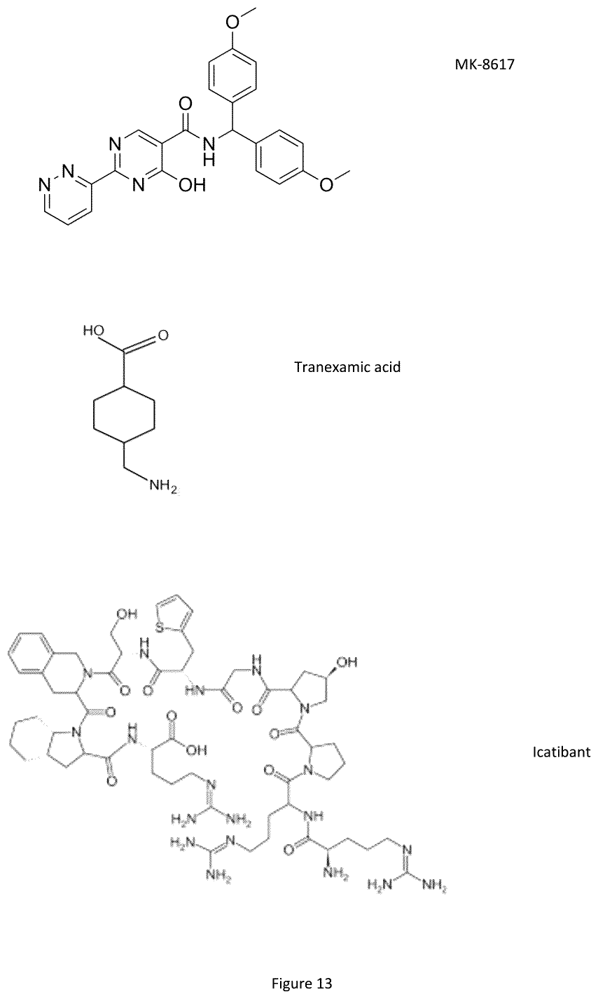

In the development of this invention, certain tests were performed in rat models. These are summarized as follows:
1) Rat Model: Rat models have been established previously that recapitulates the complex pathophysiologic changes of human with severe trauma and hemorrhagic shock (71, 72, 109, 110). The acute pathophysiologic changes includes: hypovolemic shock; acute traumatic coagulopathy (prolongation of PT, aPTT, reduction of clot firmness and platelet function); hyperfibrinolysis (rising tPA, active plasmin and D-dimer); lactatemia; immune/inflammatory response (rising cytokines), mitochondria dysfunction (decreased respiration and oxidative burst capacity in platelets), and multiple organ failure (acute kidney injury, acute lung injury).
2) Whole Blood Resuscitation in Rat Model: The outcome of using limited volume of whole blood limited resuscitation (50% of blood loss, or 20% of estimated blood volume, which is equivalent to 2 units of whole blood in human) has been previously demonstrated. Both fresh whole blood and stored whole blood (refrigerated for 7 days) transfusion at 60 min after trauma restored hemodynamics but not hemostasis measured by PT, aPTT and platelet aggregation as compared with non-resuscitation or resuscitation with same volume of Lactated Ringer's fluid. Limited resuscitation of whole blood partially attenuated lactate levels but not BUN and creatinine significantly.
3) Effect of tranexamic acid (TXA) on Fibrinolysis and Vascular Permeability: A single dose of TXA (10 mg/kg) given prior to trauma delayed elevation of prothrombin time (PT) at 30 min after trauma (FIG. 11A). At 120 min after trauma, PT significantly elevated in both groups of TXA and vehicle. Single dose of TXA (10 mg/kg) just after trauma and 45 min after the end of trauma reduces fluid movement into the lung (ALI) after polytrauma and hemorrhage in rats (FIG. 11B). In the rats with FWB resuscitation at 60 min after trauma, administration of TXA 15 min prior to resuscitation significantly reduced platelet and leukocyte infiltration and the levels of C5a and neutrophil elastase in the lung as compared with FWB (FIG. 11C).
4) PHDi and Lactate Level: In a pilot study, it was found that application of PHDi attenuated the rise of lactate in rats with polytrauma with hemorrhagic shock (FIG. 5). PHDi (MK-8617, 500 microgram per rat) was orally applied prior to trauma, and the lactate (measured by iSTAT) was measured at baseline, 2 and 4 hours after trauma. As compared with the vehicle, the average levels of lactate were attenuated about 30% at 2 and 4 hr after trauma, suggesting PHDi may be effective to improve lactate recycling under the condition of trauma and hemorrhagic shock.
5) Bradykinin receptor antagonist and vascular permeability: It was found that application of bradykinin receptor antagonist (HOE140) attenuated tissue wet/dry weight ratio in rats (polytrauma with 40% hemorrhage) (FIG. 12). HOE140 (0.4 mg/kg) was administrated as a bolus intravenously at 45 min after trauma (15 min prior to Lactated Ringer's fluid resuscitation). At 1 hour after resuscitation, the rats were euthanized and the tissues were harvested for wet weight, and then transferred into the oven at 60° C. for 14 days to be weighed as dry weight. The wet/dry ratio represented the water content of the tissue. As compared with vehicle, it was found that wet/dry weight ratio was significantly declined in brain (n=4/group), which was consistent to the results from TBI model suggesting that administration of bradykinin receptor antagonist attenuated brain blood barrier permeability.
6) Quantification of cellular damage by measuring pro-apoptotic markers and mitochondrial functionality: The results demonstrate the cold stored platelets in plasma minimize accumulation of pro-apoptotic proteins compared to room temperature-stored platelets. In a preliminary study, it was also found that mitochondria function was reduced in the tissues (liver and skeletal muscle) as shown reduction of respiration and oxidation capacity at 2 hr after polytrauma and hemorrhagic shock (data not shown).

### H. Administration of Resuscitation Fluid

Usually, hypovolemic shock results from loss of circulating blood volume due to loss of blood (hemorrhagic shock), plasma, interstitial fluid or a combination. Hemorrhagic shock refers to a condition of reduced perfusion of vital organs leading to inadequate delivery of oxygen and nutrients necessary for normal tissue and cellular functions. According to the American College of Surgeons Advanced Trauma Life Support (ATLS), hemorrhagic shock is classified based on the amount of blood loss in the average 70 kg male patient. (N Hooper and T J Armstrong, Hemorrhagic Shock, https://www.ncbi.nlm.nih.gov/books/NBK470382).

Class 1: Volume loss up to 15% of total blood volume, approximately 750 mL. Heart rate is minimally elevated or normal. Typically, there is no change in blood pressure, pulse pressure, or respiratory rate; Class 2: Volume loss from 15% to 30% of total blood volume, from 750 mL to 1500 mL. Heart rate and respiratory rate become elevated (100 BPM to 120 BPM, 20 RR to 24 RR). Pulse pressure begins to narrow, but systolic blood pressure may be unchanged to slightly decreased; Class 3: Volume loss from 30% to 40% of total blood volume, from 1500 mL to 2000 mL. A significant drop in blood pressure and changes in mental status occurs. Heart rate and respiratory rate are significantly elevated (more than 120 BPM). Urine output declines. Capillary refill is delayed; Class 4: Volume loss over 40% of total blood volume. Hypotension with narrow pulse pressure (less than 25 mmHg). Tachycardia becomes more pronounced (more than 120 BPM), and mental status becomes increasingly altered. Urine output is minimal or absent. Capillary refill is delayed.

The primary treatment of severe hemorrhage or hemorrhagic shock is to find and control the source of bleeding as soon as possible. When evacuation time is shorter than one-hour, immediate evacuation to a medical facility is recommended after airway and breathing have been secured, and resuscitation fluid IV administration can be performed at the medical facility. However, when the expected evacuation time is longer than one hour, resuscitation fluid treatment starts before evacuation.

For resuscitation fluid, blood transfusion is the best solution for pre-hospital and in-hospital treatment of hemorrhagic shock, or plasma of 1:1:1 ratio of packed red blood cells, plasma, and platelets can be used if fresh whole blood is not available. However, when blood transfusion cannot be performed in the pre-hospital setting, 4 types of fluids are recommended: crystalloid solutions (e.g., lactated Ringer's solution), colloid solutions comprising human albumin, hydroxyl ethyl starch (HES), and dextran in saline, hypertonic saline, and oxygen-carrying blood substitutes. In unstable or unresponsive hemorrhagic shock. surgical treatment is mandatory as soon as possible to control the source of bleeding.

Crystalloids or balanced salt solution, e.g., lactated Ringer's solution, is safe and inexpensive. However, because it is isotonic with an osmolarity of about 270-290 mOsm/L, and equilibrates rapidly throughout the extravascular compartment, larger volumes may be required for adequate resuscitation, resulting in decreased intravascular oncotic pressure. Colloidal molecules in the colloid solutions (e.g., human albumin, hydroxyl ethyl starch (HES), and dextran) remain in the intravascular compartment and increase oncotic pressure inside the blood vessels, and thus a relatively small total volume of resuscitative fluid is required to attain hemodynamic stability compared with crystalloid solutions. A small volume of hypertonic saline (5 ml/kg NaCl 7.5%) with or without dextran may be considered as another resuscitation fluid, although its clinical outcome is not certain in some reports. In addition, oxygen-carrying blood substitutes without problems of storage, compatibility, and disease transmission that are associated with standard blood transfusion may be another choice. There are two types of oxygen-carrying blood substitutes: fluorocarbon-based synthetic oxygen carriers and stroma-free cross linked human or non-human hemoglobin products. The fluorocarbon emulsions are easy to produce, have a long shelf life, and have minimal infectious or immunogenic effects, but they require a high fraction of inspired oxygen (FiO₂). Hemoglobin-based oxygen carriers are notable for high oxygen carrying capacity, an appreciable oncotic effect, and prolonged shelf life, but they have short plasma half-life, potential renal toxicity, hypertensive effects, and the potential of immunogenic effects.

When the source of bleeding is found and controlled, resuscitation fluid such as lactated Ringer's solution can be aggressively administered up to 2000 mL to achieve normal hemodynamic parameters. However, in the case of uncontrolled bleeding with uncertain source of bleeding, aggressive fluid infusion is not recommended because it may renew internal bleeding (“pop the clot”). Massive resuscitation fluid infusion needs to be withheld until surgical intervention. In emergency when systolic blood pressure drops below 80 mmHg, radial pulse cannot be palpated or the sensorium deteriorates, and the expected evacuation time to the medical facility exceeds one-hour, aliquots of 250 ml of lactated Ringer's solution or other resuscitation fluid may be repeatedly administered with continuous monitoring of a systolic blood pressure, radial pulse and sensorium signs. (Krausz M M. Initial resuscitation of hemorrhagic shock. World J Emerg Surg. 2006 Apr. 27; 1:14.)

### I. Exemplary Embodiments

Based on the description above and Examples in the following, methods and a kit for reducing organ failure and/or improving survivability in a patient with severe hemorrhage or hemorrhagic shock are provided here.

In certain embodiments, the resuscitation fluid is fresh whole blood or plasma of 1:1:1 ratio of packed red blood cells, plasma, and platelets. In certain embodiments, the resuscitation fluid is HEXTEND® (6% Hetastarch in Lactated Electrolyte Injection). In certain embodiments, the resuscitation fluid is a colloid solution comprising albumin 5-25% in saline, in particular albumin 25%, which is administered by continuous drip at a rate of 100 mL an hour IV. In certain embodiments, albumin solution further comprises fibrinogen. Administration of 25% albumin alone may lead to dilution effect and hypofibrinogenemia that may enhance coagulopathy. The amount of fibrinogen is calculated by the total amount lost from the hemorrhage, and the 25% albumin/fibrinogen are applied to bundle treatments of single invention and combined single invention.

In certain embodiments, the resuscitation fluid is co-administered with at least one composition comprising an anti-hemorrhage agent. The anti-hemorrhage agent is one selected from prolyl hydroxylase domain inhibitor (PHDi), antifibrinolytic agent, bradykinin receptor antagonist, and more than one agent can be co-administered, before, at the same time, and/or after resuscitation fluid infusion.

Although the main objective of this invention is pre-hospital use in the field, it can also be used in hospital settings, for example for surgery.

In certain embodiments, the composition comprising PHDi is administered is typically administered quickly after onset of hemorrhage, typically 5 min to 6 hr after onset of hemorrhage or administration of resuscitation fluid. The PHDi is one selected from a group consisting of roxadustat (FG-4592), daprodustat (GSK-1278863), vadadustat (AKB-6548), molidustat (BAY 85-3934), enarodustat (JTZ-951), and MK-8617. In certain embodiments, the composition comprising PHDi is in a solid, semi-solid, or liquid dosage form, and orally administered. In certain embodiments, the composition comprising PHDi is in a liquid dosage form and intravenously administered in bolus or drip. Although a single dose is preferred, multiple doses can be administered if need be.

In certain embodiments, the composition comprising antifibrinolytic agent is administered is typically administered quickly after onset of hemorrhage, typically 5 min to 6 hr after onset of hemorrhage or resuscitation fluid administration. The antifibrinolytic agent is one selected from a group consisting tranexamic acid, aminocaproic acid, and aprotinin. In some embodiments, tranexamic acid is administered at a dose of 1-50 mg/kg, in particular 10-30 mg/kg. In certain embodiments, the composition comprising antifibrinolytic agent is a dosage form of solid, semi-solid, or liquid dosage form, and administered through oral, parenteral, or topical route. For example, 1 gram bolus of TXA in 100 mL of normal saline can be administered over 10 minutes (slow intravenous push) to avoid hypotension caused by rapid infusion, and a 1 gram dose may be repeated over the next 8 hours.

In certain embodiments, the composition comprising bradykinin receptor antagonist is administered is typically administered quickly after onset of hemorrhage, typically 5 min to 6 hr after onset of hemorrhage or resuscitation fluid. The bradykinin receptor antagonist is bradykinin B2 receptor antagonist, icatibant. In some embodiments, icatibant is administered at a dose of 0.1-5.0 mg/kg, in particular 0.4-1.0 mg/kg. In certain embodiments, the composition comprising icatibant is in a dosage form of injectable solution for parenteral route administration, e.g., subcutaneous (SC) injection in the abdominal area. Additional doses may be administered at intervals of at least 6 hours if response is inadequate or if symptoms recur.

In certain embodiments, the calcium supplement is co-administered with resuscitation solution and an anti-hemorrhage agent or combination thereof. Calcium supplement can be calcium chloride or calcium gluconate. For mild and moderate hemorrhage, calcium carbonate, calcium citrate, calcium lactate can also be considered. Supplementation of Ca²⁺ is based on maintaining serum ionized calcium concentration at least less than 0.9 mM, as a threshold of Ca²⁺ at less than 0.6-0.7 mM is considered adequate for clot formation (80).

The anti-hemorrhage agents can be administered in combination. For example, in a certain embodiment, MK-8617 can be administered orally before resuscitation fluid infusion and TXA and/or icatibant IV/SC administered after resuscitation fluid infusion. In another embodiment, TXA and icatibant can be administered before resuscitation fluid infusion and PHDi after the resuscitation fluid infusion. In another embodiment, all three anti-hemorrhage agents can be administered before resuscitation fluid or all three after the resuscitation fluid. In another embodiment, only PHDi, TXA, or icatibant can be administered multiple times before, at, and/or after resuscitation fluid infusion.

Further, a kit is provided for pre-hospital administration of a composition/compositions to reduce organ failure and/or improve survivability in a patient with severe hemorrhage or hemorrhagic shock, comprising; at least one composition comprising a therapeutically effective amount of anti-hemorrhage agent for one time- or multiple time administration, wherein the composition is in a liquid dosage form contained in an injectable container of 5-25 mL volume (e.g., pre-filled syringe or autoinjector), or solid, semi-solid, or liquid dosage form for oral administration. The kit may further comprise non-flammable hand sanitizer, at least one pair of disposable nonlatex gloves, sterile saline for wound washing, and sterilized cleaning pads.

The anti-hemorrhagic agent is prolyl hydroxylase domain inhibitor (PHDi), antifibrinolytic agent, bradykinin receptor antagonist, or combination thereof described above, which can be co-administered, and the composition may further comprise pharmaceutically suitable excipients described in Definitions in addition to the active ingredient. The kit may further comprise albumin solution of concentration range 5-50%, in particular 25%, and the albumin solution may further comprise fibrinogen.

In case of acute hypotension or cardiac arrest due to hypovolemic/hemorrhage shock, norepinephrine 8-12 μg/min IV infusion can be co-administered with resuscitation fluid and/or anti-hemorrhage agent(s). Epinephrine or dobutamine can also be considered for such conditions. In addition to catecholamines, vasopressin (=antidiuretic hormone) bolus of doses ranging from 0.1-0.5 U/kg can also be co-administered. Norepinephrin, epinephrin and dobutamine are inotropic and open calcium channels in the heart and vascular system to increase heart contractility and blood pressure. Calcium is an ion whose influx is tightly regulated through voltage-gated- or ligand-gated channels, and there is almost no leaking like sodium or potassium. Therefore, although in high extracellular calcium concentration induced by calcium supplement, which also promote blot clot formation, in order to enhance its cardiovascular effect, calcium channel opener-ionotropic agent may be co-administered with the anti-hemorrhage agents of this disclosure in hemorrhagic shock.

In summary, the antihemorrhagic agents or combination thereof disclosed here can reduce organ damages due to hypoxia and acidosis, which is caused by increased intracellular and extracellular lactic acid. PHDi will promote HIF-1 alpha activity, which activates Cori-cycle to reduce lactate level in the blood, and bradykinin receptor inhibition will reduce vascular permeability that is also caused by cell/tissue acidosis and exacerbates blood volume loss. In addition, an antifibrinolytic agent will reduce the breakdown of blood coagulation and consequently will reduce bleeding. Therefore, by co-administering these agents with a volume expander such as 25% albumin colloid solution, they will increase survivability after severe hemorrhage or hypovolemic shock. Further, administering calcium will promote blood coagulation, and in case of severe hypotension and/or heart failure, if given together with inotropic agents, calcium will enhance vascular constriction, which will reduce vascular permeability and further volume loss, and cardiac contractility.

### Examples

### A. Methods

1. Rat models

The rat model previously reported from our lab creates the condition of trauma-induced coagulopathy. This rat model also recapitulates the complex pathophysiology of trauma with severe hemorrhagic shock.

Model-1, acute sub-lethal hemorrhagic shock with polytrauma (40% hemorrhage, fast speed hemorrhage at >1 ml/min, target MAP at 35-40 mmHg for 30 min): This model has intensive tissue damage and transient compensated hemorrhagic shock, and the animals survived for 4 hours after trauma without resuscitation or any intervention. The death occurs after 4 hours after trauma.

Briefly, Sprague-Dawley rats (350-450 gram) were anesthetized with isoflurane/100% oxygen through a nosecone and allowed to breathe spontaneously. Cannulas were placed in the left femoral artery and vein for measurement of arterial blood pressure and to obtain vascular access (blood draw and resuscitation and drug administration) respectively. Polytrauma was induced by laparotomy, crush injury to the small intestines, the left and medial liver lobes, and the right leg skeletal muscle and by fracture of the right femur. In detail, a midline incision was made through the abdominal skin and underlying muscle layers. A 10-cm section of small intestines was isolated just anterior to the cecum and run gently through a clamp covered with silastic tubing with a 2-mm distance between the clamps to cause a gentle crush injury. The right and medial lobes of the liver will receive three crushes to each lobe using the same clamp. The intestines and liver were replaced, and the abdominal incision was closed with sutures in two layers. To fracture the left femur, a modification of a 3-point impact device was used by dropping six stainless-steel balls (65 g each) stacked together, from 36 inches through a guide tube (1 inch internal diameter) to impact on a rounded aluminum blade resting on the mid-right femur with the right leg resting on two aluminum stands, one under the hip and one under the knee. A large hemostat (5 in tongs) was used to clamp the muscle of the right leg 10 times. The crush injury was intended to cause microvascular damage, as opposed to cutting, chopping, or mincing of the tissue, which would have led to uncontrolled hemorrhage. The polytrauma was performed about 10 min followed immediately by hemorrhage. Blood volume was estimated as 6% of body weight+0.77. The rats were bled to lower mean arterial pressure to 40 mmHg within 5 min and maintained at 40 mmHg until 40% of estimated blood volume is removed. Hemorrhage is usually completed within 30 min. Hemorrhage was then discontinued, and blood pressure and heart rate will be allowed to freely compensate.

Model-2, acute lethal hemorrhagic shock (mild tissue injury from cannulation, 60% hemorrhage, and fast speed hemorrhage at >1 ml/min, target MAP at 25-30 mmHg for 30 min): This model leads to acute hemorrhagic shock, and the death occurs within one hour after shock without resuscitation or any intervention.

For purposes of this disclosure, an acute lethal model was created with a modification of current sub-lethal model. The acute lethal model removes 60% of estimated blood volume and maintains MAP at 25-30 mmHg instead of 40 mmHg described in sub-lethal model. The modified model did not involve massive tissue damage in order to facilitate screen anti-shock drug to treat acute-lethal hemorrhagic shock alone.

2. End points

Model-1 (sub-lethal): Multiple organ failure is a primary end point for testing the outcome of anti-shock intervention in survivable animals with polytrauma/hemorrhagic shock at 60 min and 4 hours after trauma.

Model-2 (acute-lethal): Mortality is a primary end point (at 60 min and over time for 4 hours after hemorrhagic shock) for testing the outcome of anti-shock intervention in acute lethal hemorrhagic shock. Multiple organ failure is a secondary end point for testing the outcome of anti-shock intervention in animals which survived 60 min and 4 hours after hemorrhagic shock.

### 3. Limited Resuscitation

The Golden Hour policy is still a valuable guideline in acute management of trauma and hemorrhagic shock, which has been followed in combat casualty care by limiting the transportation time to MTF less than 60 min. A group of animals which had survived for 60 minutes received limited volume of fresh whole blood (FWB) resuscitation (20% of estimated blood volume, equivalent to 2 units of whole blood in human). The outcome of the survival animals for 4 hours with or without FWB resuscitation was measured.

The fresh whole blood (FWB) was collected at baseline, 60 min and 4 hours after hemorrhagic shock in citrate phosphate dextran (CPD 1:8 dilution from blood collection bag) from donor rats under anesthesia prior to resuscitation. The FWB was transfused at 60 min from hemorrhagic shock through femoral vein by using 200 micrometer transfusion filter set at the rate of 1 ml/min. Plasma was separated from whole blood by centrifugation at 5000 g for 10 min at 4° C., and stored at −80° C., and transfusion procedure was performed using whole blood transfusion.

A limited volume of 25% albumin (5% of estimated blood, which is one fourth of the volume calculated for limited FWB resuscitation) was applied as an anti-shock intervention, and the outcome of which was compared with that of fresh plasma and FWB. The outcome of 25% albumin in combination with single intervention was measured. Additionally, transfusion of shed whole blood immediately after hemorrhagic shock was used as a positive control for all proposed experiments.

Anti-shock drug was applied through femoral vein immediately after trauma and hemorrhagic shock: PHDi (MK-8617, 500 microgram in 500 microliter with 5% DMSO, 10% PEG400, and 85% normal saline), TXA (20 mg/kg in normal saline), Icatibant (0.5 mg/kg in normal saline), and calcium (10% calcium gluconate, 0.1 ml/kg) were reconstituted into 500 microliter solution for injection; and 25% albumin (5% of estimated blood volume) was used directly. A similar volume of vehicle (5% DMSO/10% PEG400/85% normal saline for PHDi and normal saline for TXA, Icatibant, calcium gluconate, and 25% albumin) was applied in control groups.

### 4. Clinical Laboratory Chemistry Measurements

The disclosed anti-shock intervention is expected to affect the blood level of lactate, potassium, pH, free calcium ion, creatinine, BUN and blood oxygen saturation, which are measured in the blood samples from baseline, 60 min and 4 hours after hemorrhagic shock.

All tests were performed in duplicate at the U.S. Army Institute of Surgical Research at Joint Base San Antonio/FT Sam Houston according to routine protocols. Whole blood cell count was determined by cell counter (Coulter Ac-T diff2 hematology system, Beckman Coulter, Brea, CA). Blood pH, sodium (mM), potassium (mM), chloride (mM), glucose (mg/dL), pCO₂ mmHg), pO₂ (mmHg), SaO₂ (%), HCO₃ (mmol/l), base deficient, glucose (mg/dl), creatinine (mg/dl), BUN (mg/dl), lactate (mmol/l) were measured by using an i-STAT (Abbot Labs, Abbott Park, Ill.). Platelets aggregation capacity were determined by Multiplate impedance aggregometer (Multiplate 5.0 Analyzer, Dynabyte Medical, Munich, Germany) or light transmission aggregometer (Chrono-Log) with adenosine diphosphate (ADP), collagen, and thrombin receptor activating peptide (TRAP) as agonists. Prothrombin Time (PT), Activated Partial thromboplastin Time (aPTT), and fibrinogen will be measured through ST4 (Diagnostica Stago, Parsippany, NJ). Rotational thrombo-elastometry (ROTEM, Munich, Germany) was performed to measure clot kinetics, clot firmness, and clot lysis. Tissue factor/calcium (initiate clot cascade), cytochalasin D (inhibit platelet function) and aprotinin (inhibit fibrinolysis) will be used for EXTEM, FIBTEM and APTEM assay respectively.

### 5. Multiple Organ Failure

Severe trauma and hemorrhagic shock can elicit a systemic cascade as a major precipitating cause to induce multiple organ failure (112). The incidence rate of acute lung injury (ALI) and acute kidney injury (AKI) are 33% and 34% respectively among severely injured military population which is associated with hospitalized mortality and affects long-term outcome (113, 114). For the animals that survive trauma and hemorrhagic shock, improvement of long-term outcome is dependent on whether early intervention can prevent or attenuate multiple organ failure prior or after resuscitation. Acute kidney injury (elevation of BUN and creatinine) and acute lung injury (elevation of wet/dry ratio, increased platelet/leukocyte infiltration, and elevation of MPO and neutrophil elastase) after trauma/hemorrhagic shock was previously reported. However, multiple biomarkers have been recently studied for early diagnosis of multiple organ failure in clinical patients with trauma and hemorrhagic shock (115).

The circulatory levels of Clara cell protein 16 (CCP16) and Club cell secretary protein 16 (CC16) (116-119), neutrophil gelatinase-associated lipocalin (NGAL) (120, 121), intestinal type fatty acid binding protein (I-FABP) (122-125), liver-type FABP (L-FABP) (122-125) are associated with functional damage in lung, kidney, intestine and liver respectively.

Additionally, trauma and ischemia lead to an increase in vascular permeability due to damage in endothelial integrity and function (endotheliopathy), which is associated with disruption of the glycocalyx. Syndecan-1 is a cleaved product from glycocalyx degradation, and has been one of the most sensitive biomarkers for endothelium damage and also correlates with development of multiple organ failure (115, 126).

Other markers are associated with tissue damages: sphingosine-a phosphate (SIP) is a central regulator of vascular permeability; MMP-13 is associated with inflammation and extracellular matrix degradation; Claudin-5 is a part of endothelial tight junctions to regulate blood-brain barrier function, Angiopoietin-2 is released by activated endothelium and sensitizes the endothelial cell in autocrine fashion for inflammatory stimuli; and mucin-2 is derived from the intestinal mucosa in response to intestinal permeability.

ELISA kits for Rat NGAL, I-FABP, L-FABP, Syndercan-1, MMP-13, and Claudin-5 are commercially available. The pathophysiologic changes in histology and specific biomarkers by immunohistochemistry were also quantified in tissue samples harvested at the end of experiments.

### 5.1. Acute Lung Injury Assessment:

The lung injury was assessed by respiratory, systemic and tissue biology. The respiratory function was determined by PaO₂/FiO₂ ratio which was measured by iSTAT in the whole blood sample collected from artery. Systemic and tissue biology were assessed in both plasma and lung tissue using ELISA, histology and immunohistochemistry study. Briefly, the whole lung was collected at the end of the experiment. The whole right interior lobe was collected for wet weight immediately, and kept in 600C oven for 2 weeks in order to measure dry weight. The wet/dry weight ratio was then be calculated. The whole left lobe of lung was saved for histology. The lung morphology was assessed by H&E staining and associated biomarkers were measured by immunohistochemistry. The rest of lung tissues was collected and snap-frozen, and stored at −80° C. The histologic evidence will be measured as follows: leukocyte and platelet infiltration, increase in thickness of the alveolar wall, evidence of thrombosis and hemorrhage. The levels of myeloperoxidase, complement, neutrophil elastase, matrix metalloproteinases, and cytokines/chemokines will be measured in both plasma and lung tissue homogenate. The broncho-alveolar lavage will be collected at the end of experiment to measure total protein content.

### 5.2. Acute Kidney Injury Assessment:

The BUN and creatinine was measured in blood samples at baseline and temporal points after trauma and hemorrhage. The kidney was also collected at the end of experiment for histology study to detect pathologic alternation, such as tubular degeneration, necrosis, congestion, and the evidence of thrombosis and hemorrhage. The circulatory biomarkers, neutrophil gelatinase-associated lipocalin (NGAL) were measured by ELISA in plasma at different time points.

### 5.3. Others

The liver dysfunction was measured by circulatory biomarker liver-type FABP (L-FABP, ELISA measurement) and enzyme alteration such as alanine aminotransferase (ALT) and aspartate amino transferase (AST). The functional damage in small intestine was measured by circulatory biomarker intestinal-type fatty acid binding protein (I-FABP, ELISA measurement). The wet/dry weight ratio of the intestine was also measured to present the change of vascular permeability and increase water content in the intestine. Sydercan-1 was measured by ELISA in plasma for glycocalyx degradation as a biomarkers for endothelium damage.

5.4. Other Parameters and Biomarkers that are Correlated to End Organ Function

Hemodynamics: The mean arterial blood pressure (MAP), systolic pressure, and heart rate were monitored and recorded during entire period of experiment.

Coagulopathy: coagulopathy, hypothermia, and acidosis are a bloody vicious cycle in severe trauma and hemorrhagic shock. Acute traumatic coagulopathy has been demonstrated in this animal model as characterized by prolongation of prothrombin time (PT) and activated partial thromboplastin time (aPTT), decreased fibrinogen level, reduced clot firmness and reduced platelet function. Attenuation of coagulopathy will improve hemostasis, reduce blood loss and thus attenuate the intensity of hemorrhagic shock. PT, aPTT, fibrinogen, coagulation properties, and platelet function are measured in blood samples from baseline prior to trauma, 60 min, and 4 hours after trauma.

### 6. Immune and Inflammatory Response Assessment

The anti-shock interventions are expected to down-regulate the exacerbated acute immune and inflammatory response after trauma and hemorrhagic shock. Complements, inflammatory cytokines, and chemokines in the plasma and tissue are measured using commercially available assays.

Analyses of cytokines (IL-la, IL-1(3, IL-2, IL-3, IL-4, IL-5, IL-6, IL-10, IL12p40, IL12p70, IFN-Y, TNF-α, MIP-a, RANTES, G-CSF, and GM-CSF) were performed in both plasma and tissue homogenate by multiplex cytokine assay kit (Bio-Plex Pro rat cytokine assay, Bio-Rad). The active complements in the plasma was measured by ELISA.

### 7. Histology and Immunohistochemistry:

The tissues (lung, kidney, liver, intestine, and skeletal muscle) were collected at end of experiment. The tissues sample were immerged in 4% paraformaldehyde overnight and changed to 20% sucrose overnight at 4° C. The tissues were then be processed for cryosection using OCT (Tissue-Tek, Sakura Finetek, USA). The regular Hematoxylin & Eosin staining was used for morphology observation.

The biomarkers were identified by staining with specific antibodies. Briefly, slides were fixed with 4% paraformaldehyde for 10 minutes at room temperature. Next, slides were washed with PBS containing 0.3% Triton X-100 (Sigma-Aldrich) and subjected to blocking solution of 10% donkey serum for 30 minutes at room temperature. The slide was then be treated with primary antibody and incubated overnight at 4° C., followed by PBS wash and incubation with either an Alexa Fluor 488 or Alexa Fluor 594 donkey anti-mouse or rabbit secondary antibody (species matched according to primary antibody species used) for 1 hour at room temperature. DAPI nuclear dye was applied for 15 minutes at room temperature. Slides were then observed and taken images using fluorescence microscope. The images were analyzed and quantified by ImageJ software.

### 8. Cellular Damage

Pre-hospital anti-shock intervention is expected to improve hypoxic tolerance, reduce mitochondria damage and attenuate cellular damage during hemorrhagic shock. The cellular damage is measured by cell apoptosis and mitochondria functionality in the tissues (brain, heart, liver, kidney, lung, and small intestines).

### 8.1. Mitochondrial Functionality

Mitochondrial function assay was performed from the fresh tissues (brain, heart, liver, kidney, lung and intestine) using OROBOROS high-resolution respirometry followed by manufactory protocol. In brief, about 5 mg fresh tissues were manually separated into small pieces (single fiber for muscles), and transferred into test well. The substrates, ADP, succinate, olygomycin, FCCP, rotenone and antimycin-A were serially injected to measure ATP production, maximum phosphorylated state, maximal respiration rate, and residual oxygen consumption respectively. The result was automatically normalized by tissue weight.

Mitochondrial function assay was also performed in the blood cells (platelets) using an Agilent Seahorse Extracellular Flux (XFe) analyzer. In brief, 1×10⁷ cells were plated onto Cell-Tak coated 24-well format XF plates in XF assay medium. Following calibration, the cell plate was placed into the XFe analyzer proceeded by the assay protocol. The mitochondrial stress test (Mito Stress Test) was performed by measuring the basal oxygen consumption rate (OCR) followed by sequential injection of oligomycin, FCCP and rotenone/antimycin A. The Agilent Seahorse XF Cell Mito Stress test measures key parameters of mitochondrial function by directly measuring the oxygen consumption rate (OCR) of cells. Sequential compound injections measure basal respiration, ATP production, maximal respiration, spare respiratory capacity and proton leak. The Mito Stress test utilizes modulators of respiration that target components of the electron transport chain (ETC) in the mitochondria to evaluate key parameters of metabolic function. Oligomycin, FCCP and a mixture of rotenone and antimycin- A were serially injected to measure ATP production, maximal respiration and nonmitochondrial respiration, respectively.

### 8.2. Cellular Apoptosis

Measure the intracellular pro-apoptotic markers (Bcl-xl, Bax, Bak) from the protein extracted from various tissues (brain, heart, liver, kidney, lung and intestine) using western blot. Briefly, 50 mg tissues were homogenized in ice-cold phosphate buffered saline (PBS) and lysed with RIPA buffer. The tissue lysates were then be centrifuged at 10,000×g for 10 min at 40° C. The supernatants were harvested and analyzed for protein content using bicinchoninic acid protein assay kit (BCA, Pierce, Rockford, IL, USA). To prepare samples for electrophoresis, equal amounts of protein per sample were mixed with sample buffer, heated at 100 degrees C. for 5 min, and then separated by SDS-PAGE using a mini-gel system (Bio-RAD, CA, USA). Separated proteins were then electrophoretically transferred onto polyvinylidene difluoride (PVDF) membranes using a mini blot system (Bio-Rad). Blots were then blocked for 4 hours at RT with Tris-buffered saline containing 5% nonfat dry milk, washed with TBST, and incubated overnight at 40° C. with polyclonal antibodies. The next day, blots were washed with TBSA, incubated for 1 hour at RT with appropriate IRDye secondary antibodies in TBST containing 1% nonfat dry milk. Upon washing with TBST, immunoreactive bands were detected by Odyssey LICOR detection system. Densitometry was used to determine relative protein levels. Samples were normalized within each lane to beta-actin level as an internal control.

Apoptosis in the tissue was also verified by caspase assay from the protein extracted from various tissues. Briefly, caspase-3 substrate, Asp-Glu-Val-Asp (DEVD)-7-amido-4-methylcoumarin (AMC) was purchased from Calbiochem (La Jolla, CA). Caspase-3 activity was examined by measurement of the rate of cleavage of fluorescence-conjugated substrate DEVD-AMC. 200 μg cytosolic fraction was placed onto a 96-well plate, DEVD-AMC was added in the final reaction volume of 150 ul in reaction buffer (HEPES (pH7.4) 100 mM, glycerol 20%, phenylmethylsulfonyl fluoride 0.5 mM, aprotinin 5 μg/ml. leupeptin 10 μg/ml, pepstatin 5 μg/ml, and the concentration of substrate was adjusted to 50 M. The mixture was incubated at 37° C. for 10 min in the dark. The activity was measured every 10 min up to 30 min with a fluoroplate reader and divided by time. The specificity of the assay was confirmed by addition of specific inhibitor Ac-DEVD aldehyde (DEVD-CHO) in the reaction mixture at a concentration of 50 μM during incubation.

The apoptotic cells in tissues (brain, heart, liver, kidney, lung and intestine) were identified by TUNEL staining according to manufacturer instructions (Oncor, Gaithersburg, MD). Briefly, frozen tissues sections were fixed in methanol and aceton (1:1) for 10 min at room temperature. After being washed with PBS, slides were postfixed in ethanol-acetic acid (2:1) for 5 min at −20° C. Samples were then treated with terminal deoxynucleotide transferase. After the reaction was stopped, the fragmented DNA was be visualized with fluoresceine-conjugated antibody. Nuclei were counterstained with 2,4-diamidino-2-phenylindole dihydrochloride n-bydrate (DAPI, 0.5 mg/ml).

### 9. Hypoxia Signaling Pathway

One of the key causes of cell death and organ failure during acute lethal irreversible hemorrhagic shock is hypoxia induced cellular damage. Low oxygen conditions activate the hypoxia signaling pathway primarily via the hypoxia inducible factor (HIF). Under hypoxia either due to ischemia or anemia caused by hemorrhagic shock, HIF targeted genes mediate multiple biological functions including angiogenesis apoptosis, coagulation, DNA damage and repair, cell proliferation, and metabolism, which may ultimately regulate systemic tolerance and adaptation to hypoxia, and determine the fate of cell survival and death under prolonged hypovolemic condition. Multiple HIF associated genes expression and pathways are measured in various tissue samples (brain, heart, liver, kidney, lung, and small intestines) by Hypoxia Signaling Pathway PCR Array. The results allow for an understanding of the interaction of the specific genes and identify sensitive biomarkers in response to prolonged hemorrhagic shock and anti-shock treatment.

The hypoxic-inducible factor targeted genes were measured by Hypoxia Signaling Pathway RT2 profiler PCR Array (for rat, QIAGEN). Briefly, total RNA from various tissues (brain, heart, liver, kidney, lung and intestine) was extracted by using RNeasy Kit (QIGEN, Valencia, CA) according to manufacturer instructions. Briefly, 50 mg of frozen tissue are mixed with lysis buffer and homogenized completely. The suspension is centrifuged, and 70% ethanol is added to the supernatant. The mixture is placed in a spin column, with the RNA fraction binding to the gel. The spin column is then washed with low and high salt buffer, and then eluted with nucleic acid free water. RNA concentration is determined with spectrophotometer. The RNA is then be converted to cDNA by using RT2 HT First Strand Kit (QIAGEN). The cDNA is mixed with the appropriate ready-to-use PCR mastermix from the array kit, aliquot equal volumes to each well of the same plate, and then run the real-time PCR cycling program (ROCHE LightCycler).

### 10. Intracellular ATP:

Various tissues were taken at the end of the experiment, placed on ice and immediately minced finely with a razor blade. Approximately 100 ul of minced slurry was immediately extracted as follows: Di- and Tri phosphates were extracted using 1 ml of 50% MeOH, 50% 1 M ammonium formate, centrifuged (20,000 g for 10 min), and the supernatant dried by savant. Monophosphates are extracted using 1 ml of 100% EtOH, 10 mM formic acid, centrifuged (20 Kg for 10 min) and the supernatant dried by savant. All samples were brought up in 200 ul of 5% MeCN 0.1% formic acid for analysis by liquid chromatography followed by Tandem Mass Spectroscopy (LC-MS/MS, TSQ Quantiva, Thermo Scientific, Waltham, MA). The chromatography used a 3 minute ramp from 0 to 50% mobile phase B (100% MeCN, 0.1% formic acid) from mobile phase A (0.1% formic acid) for separation of the analytes on a Reverse Phase (C18) 50×2.1 mm 5u (Luna, Phenomenex, Torrance, Ca) before analysis by mass spectroscopy. Mass spectroscopy measures four to six daughters of each parent analyte after collision by argon gas.

### 11. Liquid Chromatography-Mass Spectrometry (LC-MS)-Based Metabolic Change Analysis

Liquid chromatography-mass spectrometry (LC-MS)-based metabolomics analysis is able to identify the metabolic derangements in response to trauma/hemorrhagic shock. The change of metabolomics (especially the metabolites associated with TCA cycle and glycolysis) in the various tissues (brain, heart, liver, kidney, lung, and small intestines) and plasma help to identify the sensitive markers in response to shock and anti-shock treatment. For this work, the TCA cycle intermediates such as succinate and fumarate, will be focused, which are known to accumulate in shock, as markers of efficiency for anti-shock interventions.

Various tissues were taken at the end of the experiment, placed on ice and immediately minced finely with a razor blade. Approximately 100 ul of minced slurry was immediately extracted using MeOH: H₂O: Chloroform (2:1:2 by volume), centrifuged at 20 Kg for 10 min, and supernatant filtered over 5K MW cut off (Centriprep, Millipore). The eluent is dried by Savant. All samples were brought up in 200 ul of 5% MeCN 0.1% formic acid for analysis by liquid chromatography followed by Tandem Mass Spectroscopy (LC-MS/MS, TSQ Quantiva, Thermo Scientific, Waltham, MA). The chromatography used a 3 minute ramp from 0 to 50% mobile phase B (100% MeCN, 0.1% formic acid) from mobile phase A (0.1% formic acid) for separation of the analysts on a Reverse Phase (C18) 50×2.1 mm 5u (Luna, Phenomenex, Torrance, Ca) before analysis by mass spectroscopy. Mass spectroscopy measures four to six daughters of each parent analyst after collision by argon gas.

### 12. Power Analysis

As for the primary outcome, there is no preliminary data of using single interventions of PHDi, TXA, Icatibant, or Calcium gluconate in the acute lethal model of hemorrhagic shock. However, as we predict that administration of 25% albumin will be crucial for survival in severe hypovolemic shock, the 25% albumin combined with single intervention will be a more realistic approach than single drugs for better outcomes of both mortality and organ failure in acute lethal models of trauma and hemorrhagic shock. There is no data that can be used for power analysis of primary endpoint (mortality) for proposed animal models, except for previous study showing that the mortality of using 25% albumin is 11% versus 89% of non-resuscitation in a rabbit model with trauma and hemorrhagic shock. The sample size analysis suggests that animal number at 8 per group is adequate to show a significant difference (alpha) of 0.05 and a power of 0.80 with treatment of 25% albumin as compared with control. Therefore, if using a sample size of 10 rats per group, we predict that we will reliably detect a significant difference (alpha) of 0.05 and a power of 0.80 in terms of the significant change in mortality.

As for the multiple organ failure and other biomarkers, it was shown a significant acute traumatic coagulopathy (e.g. elevation of prothrombin time), lactatemia (increased lactate), acute kidney injury (e.g. elevation of BUN, creatinine), acute lung injury (e.g. increased wet/dry weight ratio, and platelet/leukocyte infiltration) in rats with polytrauma and hemorrhagic shock based on the previous study using a sample size of 10 rats/group. For example, sample size analysis suggests that the significant difference (alpha) of 0.05 with a power of 0.80 is expected to be shown using 6 rats for PT, 5 rats for creatinine (acute kidney injury), 7 rats for wet/dry ratio (acute lung injury), and 4 rats for lactate at 2 hours after trauma as compared with the baseline, suggesting that adequate power should be generated from the size 7-10 rats/groups for most of the variables in secondary outcomes in the models that we will be testing.

Based on the pilot study of administration of PHD in trauma/hemorrhagic shock ((PHDi (n=4) versus vehicle (n=3)), the lactate declined from 3.395±0.969 (Vehicle) to 2.365±0.733 (PHDi) mmol/1 at 4 hours after trauma, suggesting that animal size at 10-15/group is adequate to achieve significant difference (alpha) of 0.05 with a power of 0.80 in terms of the significant change in lactate in response to PHDi administration.

Based on the pilot study of administration of Icatibant (HOE140) in trauma/hemorrhagic shock (n=4 per group), the wet/dry weight ratio was declined in brain (from 4.735±0.024 to 4.674±0.023), traumatized skeletal muscle (from 4.491±0.112 to 4.247±0.176) and intestine (from 4.528±0.112 to 4.399±0.090), suggesting that animal size at 4 (for brain), 5-10 (for traumatized muscle) and 9-13 (for intestine) is adequate to achieve significant difference (alpha) of 0.05 with a power of 0.80 in terms of the significant change in tissue wet/dry ratio in response to Icatibant administration.

Based on the preliminary power analysis of the key variables selected from both primary and secondary outcomes of previous and pilot studies, we will propose to use a sample size of 15 rats/group, and do an interim analysis at 10 rats/group to determine if the power is sufficient in order to detect a significant difference (alpha) of 0.05 with a power of 0.80.

### 13. Statistical Analysis

Statistical significance will be reported at appropriate confidence intervals, and all results will be analyzed according to standard statistical techniques. The treatment will be tested in an acute lethal model (mortality occurs within 60 min after trauma/hemorrhagic shock) and sub-lethal model (survival for 4 hours after trauma/hemorrhagic shock). The mortality resulting from administration of a single or bundle of interventions, will be analyzed by chi-square as compared with accordingly vehicle group, sham and positive control groups that are applied to each treatment respectively. The time of death will also be recorded so that the Kaplan-Meier curve will be generated and the survival time will be analyzed among the groups. Secondary outcomes and other parameters will be measured in blood samples collected at baseline, 60 min (minimum measurements), and 4 hours after hemorrhagic shock. Overall significance of means of multiple groups will be assessed by analysis of the variance. Data will be further analyzed by separate one-way or two-way ANOVA (Tukey's test etc.), and tests between responses from treatment and non-treatment groups will be performed by Students t-test or other analysis. A p value <0.05 will be considered significant difference.

### 14. Dose Calculation for Human Application

The starting dose of single anti-shock drug is based on the dose currently recommended in human subjects. There is no universal guideline how to convert the human dose to animal dose effectively because the drug pharmacokinetics may be varied between human and animals for a particular drug. The common acceptable concept is that smaller animals have higher metabolic rates, and physiological process of smaller animals is faster than larger animals. Therefore, smaller animals always are considered using larger treatment dose as compared with larger animals and human. However, based on our own experience and numerous studies published, the human dose is also effective in smaller animals such as rodent. It just the matter that the maximum effect is desired if using a higher dose in smaller animals. Based on FDA guidelines, the maximum recommended starting dose is calculated by the animal dose divided by 0.162 (for rat) so that the dose used in rat can be estimated by human dose multiplied by 6 (which is 6 times of human dose). Therefore, we will potentially use 6 times higher dosing (from human) to test the maximum effect in rats.

### B. Results

A rat model was previously established that recapitulates the complex pathophysiologic changes of human with severe trauma and hemorrhagic shock (71, 72, 109, 110). The acute pathophysiologic changes includes: hypovolemic shock; acute traumatic coagulopathy (prolongation of PT, aPTT, reduction of clot firmness and platelet function); hyperfibrinolysis (rising tPA, active plasmin and D-dimer); lactatemia; immune/inflammatory response (rising cytokines, mitochondria dysfunction (decreased respiration and oxidative burst capacity in platelets), multiple organ failure (acute kidney injury, and acute lung injury).

Further, quantification of cellular damage was performed by measuring pro-apoptotic markers and mitochondrial functionality (data not shown). This study demonstrates the cold stored platelets in plasma minimize accumulation of pro-apoptotic proteins compared with room temperature-stored platelets.

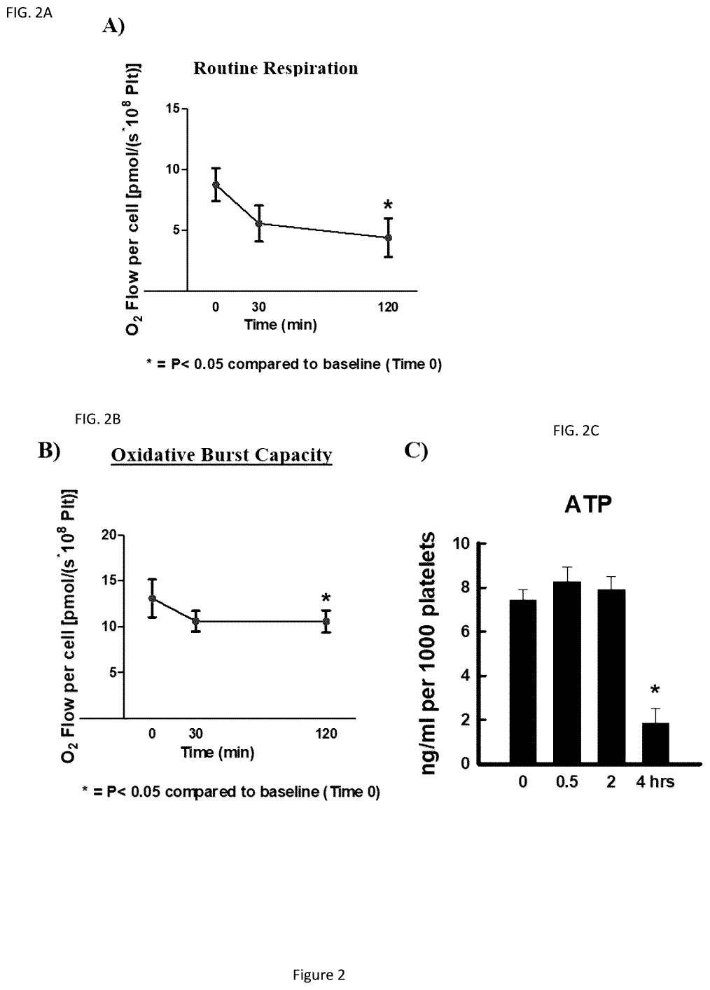

In a preliminary study, mitochondrial respiration, maximal oxygen utilization, and individual mitochondrial complex dependent respiration were assessed with high-resolution respirometry. A significant decrease in routine respiration and oxidative burst capacity were measured for 120 min after trauma/hemorrhage and compared with the levels of baseline (FIG. 2A and FIG. 2B). Intracellular ATP also significantly declined 4 hours after trauma (FIG. 2C). indicating that mitochondria function was reduced in the platelets.

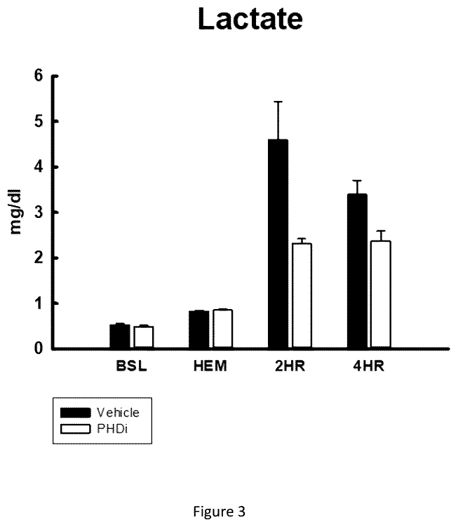

In a pilot study (FIG. 3), it was found that oral administration of PHDi (MK-8617, 1 mg/kg) prior to trauma attenuated the rise of lactate in rats with polytrauma with hemorrhagic shock. In this experiment, PHDi (MK-8617, 1 mg/mL in 500 μL composed of 5% DMSO (dimethyl sulfoxide), 10% PEG 400 (polyethylene glycol 400), and 85% normal saline, average dose at 1.26±0.09 mg/kg, n=4) or vehicle (dissolvent only, n=4) was administered orally by gavage prior to trauma/hemorrhage.

Then, isoflurane anesthetized Sprague-Dawley rats underwent polytrauma that was induced by damaging the left and medial liver lobes, the small intestines, the right leg skeletal muscles, and by fracturing the right femur. The rats were then bled to maintain mean arterial pressure (MAP) at 40 mmHg until 40% of the blood volume was removed. The surviving animals were euthanized 4 hours after trauma.

The results are that all animals survived after 4 hours except for one animal in the vehicle group that died at approximately 3-hour post-trauma. The lactate in whole blood samples were collected at baseline (BSL), shed hemorrhage blood (HEM), 2- and 4 hours after trauma, and the lactate was measured by iSTAT. As compared with the vehicle, the lactate declined from 3.395±0.969 (Vehicle) to 2.365±0.733 (PHDi) mmol/1 at 4 hours after trauma. The average levels of lactate in PHDi treated rats were attenuated by about 30% at 2- and 4 hours after trauma (FIG. 3), suggesting PHDi may be effective to improve lactate recycling under the condition of trauma and hemorrhagic shock. The results of this pilot study suggested that pre-treatment of PHDi (MK-8617) improved the outcome of trauma/hemorrhage by mitigating the elevation of lactate at 2 and 4 hours after trauma/hemorrhage.

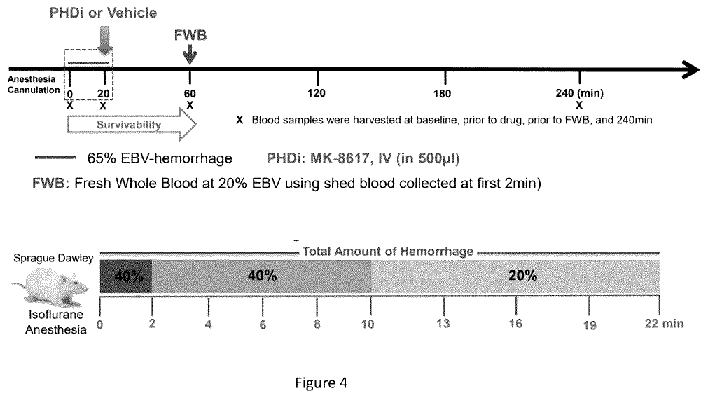

FIG. 4 illustrates a whole blood resuscitation experimental scheme in a rat model. The outcome of using limited volume of whole blood limited resuscitation (50% of blood loss, or 20% of estimated blood volume, which is equivalent to 2 units of whole blood in human) were already demonstrated in our previous publication (111). Both fresh whole blood and stored whole blood (refrigerated for 7 days) transfusion at 60 min after trauma restored hemodynamics, but no hemostasis was measured, i.e., PT, aPTT, and platelet aggregation were compared in non-resuscitation or resuscitation with the same volume of lactated Ringer's fluid. Limited resuscitation of whole blood partially attenuated lactate levels but not BUN and creatinine significantly.

For the experiments in this disclosure, fixed-volume hemorrhage (65% estimated blood volume (EBV)) was performed in isoflurane anesthetized Sprague-Dawley rats. In this model, the initial 40% of total volume of hemorrhage was performed by continuous blood draw using syringe pump within first 2 min, followed by manually repeatedly drawing 1.5 ml of whole blood every 2 min at 10 min, and 1 ml of whole blood every 3 min thereafter until achieving the targeted volume of hemorrhage, and thus 40% bleeding occurs within the first 2 minutes (fast hemorrhage), another 40% between 2-10 minutes, and the rest 20% between 10-22 minutes (slow hemorrhage).

Then, the animals were treated with 500 μL PHDi (MK-8617 at 1 mg/ml, in a dissolvent composed of 5% DMSO, 10% PEG 400, and 85% normal saline, average dose at 1.24±0.07 mg/kg) or vehicle (n=13 each) intravenously at 20 min after hemorrhage. At 60 min after hemorrhage, surviving animals were resuscitated with fresh whole blood (FWB, 20% EBV) using shed blood collected at 2 min after hemorrhage. The 60 min- or 4 hour survivability was analyzed.

For the measurement of metabolites (lactate and HCO3) and ATP, blood samples were harvested at time point 0 (baseline), 20 minutes (prior to PHDi), 60 minutes (prior to fresh whole blood (FWB)), and 240 minutes after polytrauma/hemorrhage.

As shown in FIG. 5, without any intervention, time to death was 43±3 min with 60% EBV, and 30±4 min with 65% EBV drained from the rats. (EBV: estimated blood volume. Survivability decreased and lactate level increased as EBV increased.

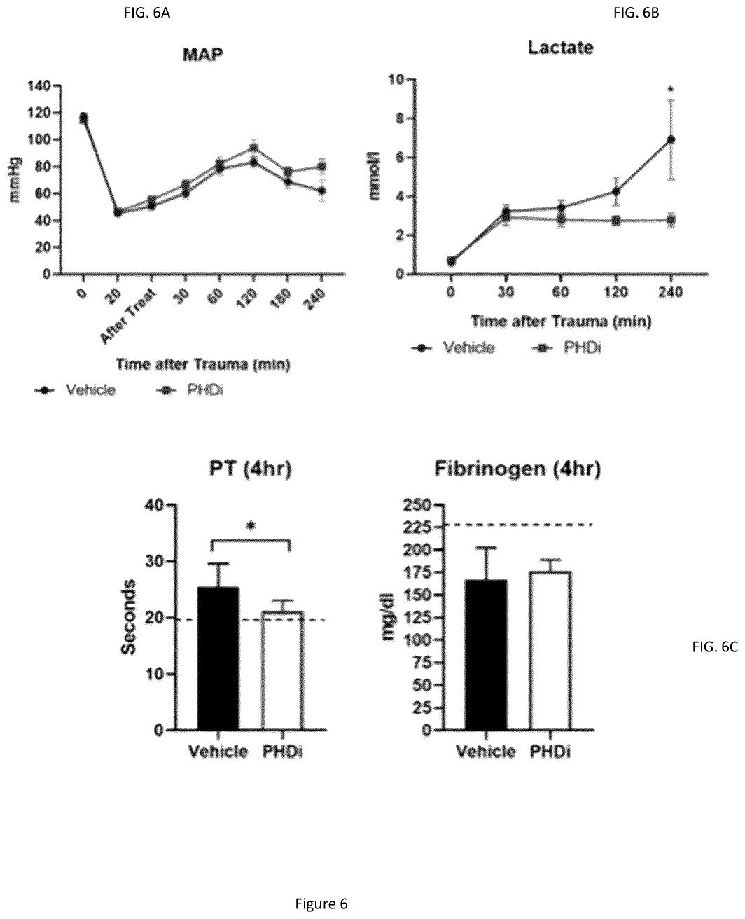

FIG. 6 shows the effect of intravenous administration of PHDi after trauma//hemorrhage in a rodent model of lethal hemorrhagic shock. PHDi (MK-8617, 1 mg/ml in 500 μL of 5% DMSO, 10% PEG 400, and 85% normal saline, average dose 1.26±0.05 mg/kg, n=8), or vehicle (n=7) was administered intravenously at 20 min after trauma. The surviving animals were euthanized 4 hours after trauma. MAP was significantly decreased after hemorrhage, but it was significantly higher in PHDi compared with vehicle after treatment. In both groups, lactate was significantly elevated at 19 min, and there was about 30% reduction in the elevation of lactate at 60 min in PHDi treated animals as compared with vehicle, indicating PHDi mitigates the elevation of lactate (hyperlactatemia). PHDi also reduced the prolongation of prothrombin time (PT, acute traumatic coagulopathy) in polytraum/hemorrhage.

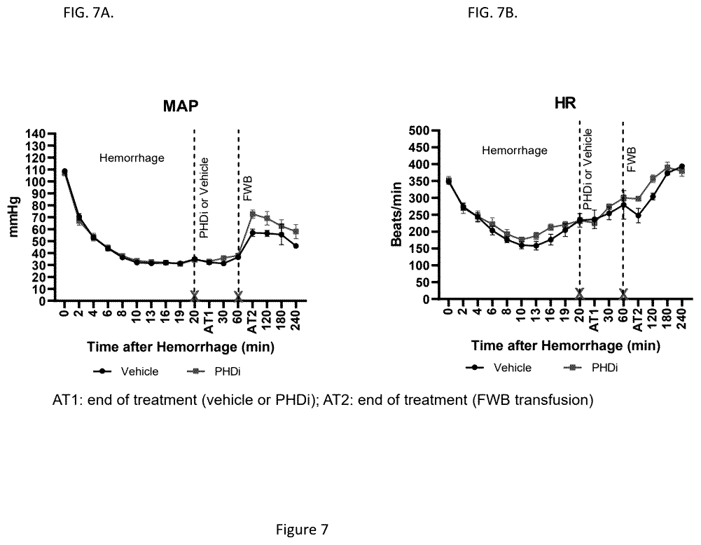

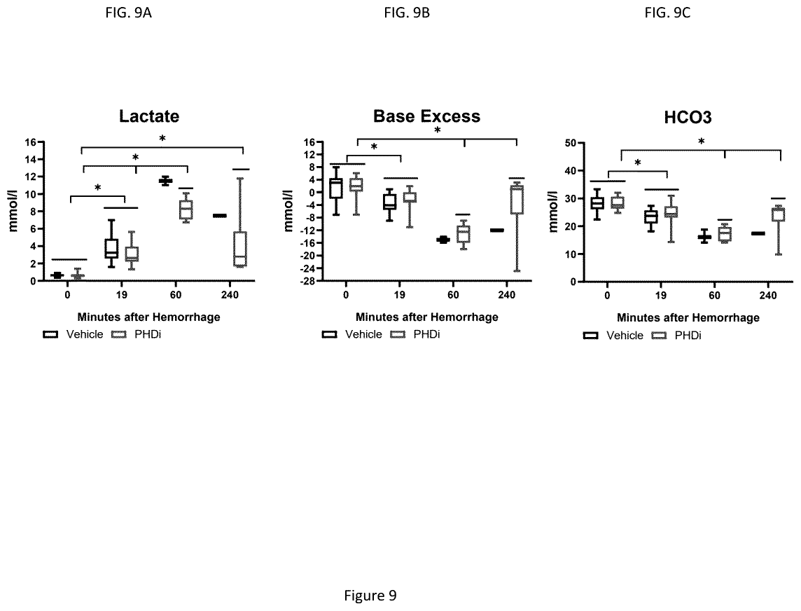

FIG. 7 shows the effect of intravenous administration of PHDi after trauma/hemorrhage on hemodynamics (MAP, HR). The experiment was performed as described with FIG. 6. In both vehicle treated control rats and PHDi treated rats, MAP was significantly decreased after hemorrhage, but it was significantly higher in PHDi treated rats compared with vehicle treated rats 10 min after treatment (35.9±1.2 (PHDi) vs. 31.3±1.1 (Vehicle) mmHg, p=0.02). In PHDi treated animals, whole blood resuscitation (FWB) led to partial restoration of MAP (FIG. 7A). In PHDi treated animals, whole blood resuscitation led to partially restored MAP and reduced lactate at 4 hr after transfusion (FIG. 9A).

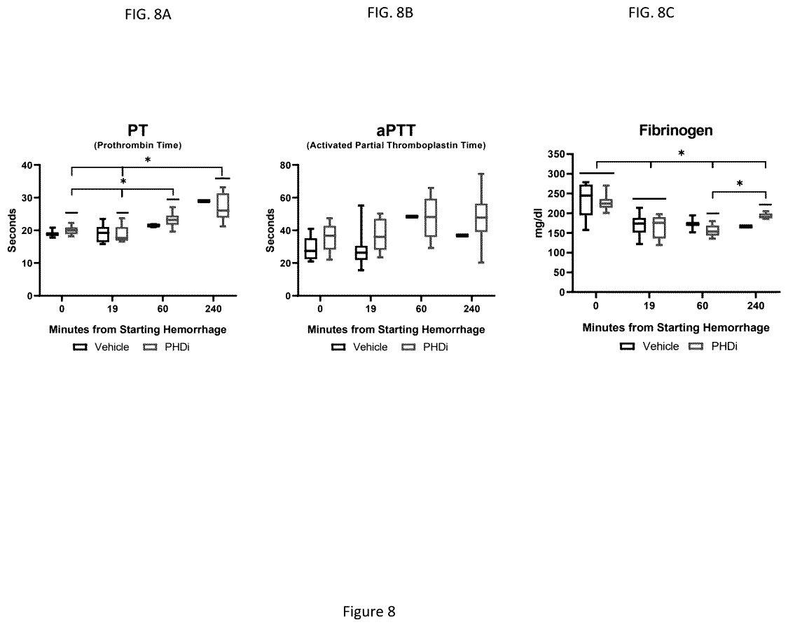

FIG. 8 shows the effect of intravenous administration of PHDi after trauma/hemorrhage on hemostasis (prothrombin time (PT), activated partial thromboplastin time (aPTT), and fibrinogen by ST4). PT was not significantly changed at the end of hemorrhage, but it was significantly elevated in surviving animals (PHDi) at 60 min and 4 hours followed by whole blood resuscitation. The similar pattern of change was observed in aPTT. The fibrinogen significantly decreased at the end of hemorrhage, and it was significantly elevated at 4 hours followed by whole blood resuscitation but still significantly higher than the level at the baseline.

FIG. 9 shows the effect of intravenous administration of PHDi after trauma/hemorrhage on metabolism (lactate by iSTAT, base excess, and HCO₃). In both groups, lactate was significantly elevated at 19 min, but there was about 30% reduction in the elevation of lactate at 60 min in PHDi treated animals as compared with vehicle. HCO3 and base excess significantly decreased at the end of hemorrhage, and it was partially elevated at 4 hours followed by whole blood resuscitation but still significantly lower than the levels at baseline

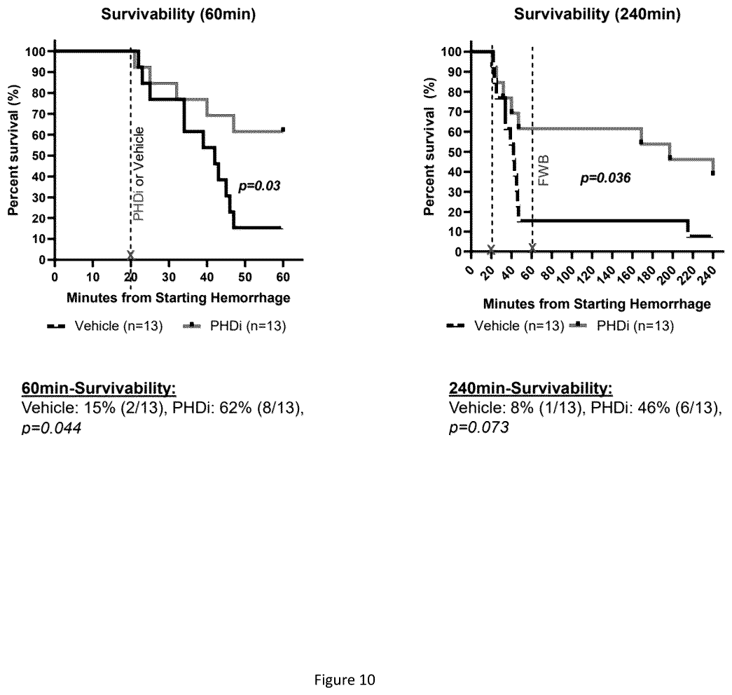

FIG. 10 shows the effect of intravenous administration of PHDi after trauma/hemorrhage on survivability (Kaplan-Meier plots with log-rank test, Fisher's exact test). There was a significant improvement in 60 min survivability of PHDi treated animals compared with vehicle (62% (PHDi) vs. 15% (vehicle), p=0.03). Among surviving animals that received whole blood resuscitation, 6 of 8 of PHDi and 1 of 2 of vehicle treated animals survived for 4 hours after hemorrhage (p=0.036). In summary, acute administration of PHDi extends the time to death in rats with lethal hemorrhagic shock. The results of this disclosure demonstrated a novel pre-hospital therapeutic towards improving outcomes in resource constrained operations with potentially prolonged evacuation time for patients experiencing severe hemorrhagic shock.

FIG. 11 shows the effect of anti-fibrinolytic agent (tranexamic acid; TXA) on fibrinolysis and vascular permeability. Single dose of TXA (10 mg/kg) was given prior to trauma, and this delayed elevation of prothrombin time (PT) at 30 min after trauma (FIG. 11A). At 120 min after trauma, PT significantly elevated in both groups of TXA and vehicle. Single dose of TXA (10 mg/kg) given just after trauma, and 45 min after the end of trauma reduce fluid movement into the lung (ALI) after polytrauma and hemorrhage in rats (73). FIG. 11B suggests TXA effect on vascular permeability. TXA was given just before trauma (BT, red), just after trauma (AT, green) and 45 min after the end of trauma (yellow). FIG. 11C shows TXA effect on acute lung injury. TXA was given 45 min after trauma (15 min prior to resuscitation). In the rats with FWB resuscitation at 60 min after trauma, administration of TXA 15 min prior to resuscitation significantly reduced the levels of platelet and leukocytes (neutrophils and monocytes) infiltration, and C5a and neutrophil elastase in the lung as compared with FWB.

FIG. 12 shows that bradykinin receptor antagonist (icatibant, HOE140) attenuated tissue wet/dry weight ratio in rats (polytrauma with 40% hemorrhage). Icatibant (HOE140), which is a selective competitive antagonist at the bradykinin type 2 (B2) receptor, is a synthetic decapeptide with a structure similar to bradykinin, but with 5 nonproteinogenic amino acids. It consists of ten amino acids in the following sequence: H-D-Arg-Arg-Pro-Hyp-Gly-Thi-Ser-D-Tic-Oic-Arg-OH. HOE140 (0.4 mg/kg in lactate Ringer's fluid) was administrated as a bolus intravenously at 45 min after trauma (15 min prior to lactated Ringer's fluid resuscitation (20% of Blood Volume)). At 1 hour after resuscitation, the rats were euthanized and the tissues were harvested for wet weight, and then transferred into the oven at 60° C. for 14 days to be weighed as dry weight. The wet/dry ratio represented the water content of the tissue. As compared to vehicle and administration of icatibant (HOE140) in trauma/hemorrhagic shock (n=4 per group), the wet/dry weight ratio was declined in brain (from 4.735±0.024 to 4.674±0.023), traumatized skeletal muscle (from 4.491±0.112 to 4.247±0.176) and intestine (from 4.528±0.112 to 4.399±0.090); i.e., wet/dry weight ratio was significantly declined in brain (n=4/group), which was consistent to the results from TBI model suggesting that administration of bradykinin receptor antagonist attenuated brain blood barrier permeability. These results indicate that tissue water content and vascular permeability were reduced due to HOE140.

### REFERENCES

1. Eastridge B J, et al. (2011) Died of Wounds on the Battlefield: Causation and Implications for Improving Combat Casualty Care. J Trauma 71:S4-S8.
2. MacLeod J B A, Cohn S M, Johnson E W, & McKenney M G (2007) Trauma deaths in the first hour: are they all unsalvageable injuries? Am J Surg 193(2):195-199.
3. Eastridge B J, et al. (2012) Death on the battlefield (2001-2011): Implications for the future of combat casualty care. J Trauma Acute Care 73:S431-S437.
4. Shackelford S A, et al. (2017) Association of Pre-hospital Blood Product Transfusion During Medical Evacuation of Combat Casualties in Afghanistan With Acute and 30-Day Survival. Jama-J Am Med Assoc 318(16):1581-1591.
5. RM G (2014) Duty: Memois of a Secretary at Wat. New York. NY: Random House:304-305.
6. Kotwal R S, et al. (2016) The Effect of a Golden Hour Policy on the Morbidity and Mortality of Combat Casualties. JAMA surgery 151(1):15-24.
7. Szopinski J, Kusza K, & Semionow M (2011) Microcirculatory Responses to Hypovolemic Shock. J Trauma 71(6):1779-1788.
8. Wang J, Liang T, Louis L, Nicolaou S, & McLaughlin P D (2013) Hypovolemic Shock Complex in the Trauma Setting: A Pictorial Review. Can Assoc Radiol J 64(2):156-163.
9. Dewar D, Moore F A, Moore E E, & Balogh Z (2009) Postinjury multiple organ failure. Injury 40(9):912-918.
10. Slaughter A L, et al. (2018) The Metabolopathy of Tissue Injury, Hemorrhagic Shock, and Resuscitation in a Rat Model. Shock 49(5):580-590.
11. Tannahill G M, et al. (2013) Succinate is an inflammatory signal that induces IL-1beta through HIF-1alpha. Nature 496(7444):238-242.
12. Chouchani E T, et al. (2014) Ischaemic accumulation of succinate controls reperfusion injury through mitochondrial ROS. Nature 515(7527):431-435.
13. Sena-Perez A, et al. (2010) Extended ischemia prevents HIF1alpha degradation at reoxygenation by impairing prolyl-hydroxylation: role of Krebs cycle metabolites. J Biol Chem 285(24): 18217-18224.
14. Isaacs J S, et al. (2005) HIF overexpression correlates with biallelic loss of fumarate hydratase in renal cancer: novel role of fumarate in regulation of HIF stability. Cancer ce118(2):143-153.
15. Koivunen P, et al. (2007) Inhibition of hypoxia-inducible factor (HIF) hydroxylases by citric acid cycle intermediates: possible links between cell metabolism and stabilization of HIF. J Biol Chem 282(7):4524-4532.
16. Chen S & Sang N (2016) Hypoxia-Inducible Factor-1: A Critical Player in the Survival Strategy of Stressed Cells. Journal of cellular biochemistry 117(2):267-278.
17. Kumar H & Choi D K (2015) Hypoxia Inducible Factor Pathway and Physiological Adaptation: A Cell Survival Pathway? Mediators of inflammation 2015:584758.
18. Odom S R, et al. (2013) Lactate clearance as a predictor of mortality in trauma patients. The journal of trauma and acute care surgery 74(4):999-1004.
19. Zhang Z & Xu X (2014) Lactate clearance is a useful biomarker for the prediction of all-cause mortality in critically ill patients: a systematic review and meta-analysis*. Crit Care Med 42(9):2118-2125.
20. Sauaia A, et al. (1994) Early Predictors of Postinjury Multiple Organ Failure. Arch Surg-Chicago 129(1):39-45.
21. Wang G L, Jiang B H, Rue E A, & Semenza G L (1995) Hypoxia-Inducible Factor-1 Is a Basic-Helix-Loop-Helix-Pas Heterodimer Regulated by Cellular 0-2 Tension. P Natl Acad Sci USA 92(12):5510-5514.
22. Wang G L & Semenza G L (1995) Purification and Characterization of Hypoxia-Inducible Factor-1. J Biol Chem 270(3):1230-1237.
23. Ohh M, et al. (2000) Ubiquitination of hypoxia-inducible factor requires direct binding to the beta-domain of the von Hippel-Lindau protein. Nat Cell Biol 2(7):423-427.
24. Ivan M, et al. (2001) HIF alpha targeted for VHL-mediated destruction by proline hydroxylation: Implications for 0-2 sensing. Science 292(5516):464-468.
25. minamishima Y A, et al. (2008) Somatic inactivation of the PHD2 prolyl hydroxylase causes polycythemia and congestive heart failure. Blood 111(6):3236-3244.
26. Jaakkola P, et al. (2001) Targeting of HIF-alpha to the von Hippel-Lindau ubiquitylation complex by 0-2-regulated prolyl hydroxylation. Science 292(5516):468-472.
27. Semenza G L, Nejfelt M K, Chi S M, & Antonarakis S E (1991) Hypoxia-Inducible Nuclear Factors Bind to an Enhancer Element Located 3′ to the Human Eryt hoursopoietin Gene. P Natl Acad Sci USA 88(13):5680-5684.
28. Semenza G L & Wang G L (1992) A Nuclear Factor Induced by Hypoxia Via Denovo Protein-Synthesis Binds to the Human Eryt hoursopoietin Gene Enhancer at a Site Required for Transcriptional Activation. Mol Cell Biol 12(12):5447-5454.
29. Papandreou I, Cairns R A, Fontana L, Lim A L, & Denko N C (2006) HIF-1 mediates adaptation to hypoxia by actively downregulating mitochondrial oxygen consumption. Cell Metab 3(3):187-197.
30. Kim J W, Tchernyshyov I, Semenza G L, & Dang C V (2006) HIF-1-mediated expression of pyruvate dehydrogenase kinase: A metabolic switch required for cellular adaptation to hypoxia. Cell Metab 3(3):177-185.
31. Fukuda R, et al. (2007) HIF-1 regulates cytoc hoursome oxidase subunits to optimize efficiency of respiration in hypoxic cells. Cell 129(1):111-122.
32. Aragones J, et al. (2008) Deficiency or inhibition of oxygen sensor Phd1 induces hypoxia tolerance by reprogramming basal metabolism. Nat Genet 40(2):170-180.
33. Kelly D P (2008) Hypoxic reprogramming. Nat Genet 40(2):132-134.
34. Zhao T, et al. (2014) HIF-1-mediated metabolic reprogramming reduces ROS levels and facilitates the metastatic colonization of cancers in lungs. Sci Rep-Uk 4.
35. Suhara T, et al. (2015) Inhibition of the oxygen sensor PHD2 in the liver improves survival in lactic acidosis by activating the Cori cycle. P Natl Acad Sci USA 112(37):11642-11647.
36. Oyaizu-Toramaru T, et al. (2017) Targeting Oxygen-Sensing Prolyl Hydroxylase for Metfor min-Associated Lactic Acidosis Treatment. Mol Cell Biol 37(16).
37. Oyaizu-Toramaru T, et al. (2017) Targeting the oxygen sensor PHD (prolyl hydroxylase) for the treatment of lethal lactic acidosis. J Pharmacol Sci 133(3):S101-S101.
38. Zhong Z, et al. (2008) Activation of the oxygen-sensing signal cascade prevents mitochondrial injury after mouse liver ischemia-reperfusion. Am J Physiol-Gastr L 295(4):G823-G832.
39. Kapitsinou P P, et al. (2014) Endothelial HIF-2 mediates protection and recovery from ischemic kidney injury. J Clin Invest 124(6):2396-2409.
40. Gong H X, et al. (2015) HIF2 alpha signaling inhibits adherens junctional disruption in acute lung injury. J Clin Invest 125(2):652-664.
41. Sawamura A, et al. (2009) Disseminated intravascular coagulation with a fibrinolytic phenotype at an early phase of trauma predicts mortality. T hoursomb Res 124(5):608-613.
42. Chapman M P, et al. (2016) Overwhelming tPA release, not PAI-1 degradation, is responsible for hyperfibrinolysis in severely injured trauma patients. The journal of trauma and acute care surgery 80(1):16-23; discussion 23-15.
43. Brohi K, Singh J, Heron M, & Coats T (2003) Acute traumatic coagulopathy. J Trauma 54(6):1127-1130.
44. Niles S E, et al. (2008) Increased mortality associated with the early coagulopathy of trauma in combat casualties. J Trauma 64(6):1459-1463.
45. Tengborn L, Blomback M, & Berntorp E (2015) Tranexamic acid—an old drug still going strong and making a revival. T hoursomb Res 135(2):231-242.
46. Ker K, Edwards P, Perel P, Shakur H, & Roberts I (2012) Effect of tranexamic acid on surgical bleeding: systematic review and cumulative meta-analysis. BMJ 344:e3054.
47. Collaborators WT (2017) Effect of early tranexamic acid administration on mortality, hysterectomy, and other morbidities in women with post-partum haemorrhage (WOMAN): an international, randomised, double-blind, placebo-controlled trial. Lancet 389(10084):2105-2116.
48. Collaborators C-t, et al. (2010) Effects of tranexamic acid on death, vascular occlusive events, and blood transfusion in trauma patients with significant haemorrhage (CRASH-2): a randomised, placebo-controlled trial. Lancet 376(9734):23-32.
49. Collaborators C-, et al. (2011) The importance of early treatment with tranexamic acid in bleeding trauma patients: an exploratory analysis of the CRASH-2 randomised controlled trial. Lancet 377(9771):1096-1101, 1101 e1091-1092.
50. Morrison J J, Dubose J J, Rasmussen T E, & Midwinter M J (2012) Military Application of Tranexamic Acid in Trauma Emergency Resuscitation (MATTERs) Study. Arch Surg 147(2):113-119.
51. Morrison J J, et al. (2013) Association of cryoprecipitate and tranexamic acid with improved survival following wartime injury: findings from the MATTERs II Study. JAMA Surg 148(3):218-225.
52. Cap A P, et al. (2011) Tranexamic acid for trauma patients: a critical review of the literature. J Trauma 71(1 Suppl):59-14.
53. Puigdellivol E, Carral M E, Moreno J, Pladelfina J M, & Jane F (1985) Pharmacokinetics and Absolute Bioavailability of Intramuscular Tranexamic Acid in Man. Int J Clin Pharm Th 23(6):298-301.
54. Vu E N W W, Yeung T C, Callaway D W (2018) Intramuscular Tranexamic Acid in Tactical and Combat Settings. Journal of Special Operations Medicine 18(1):62-68.
55. Boysen S R, et al. (2017) Comparison of tranexamic acid plasma concentrations when administered via intraosseous and intravenous routes. Am J Emerg Med 35(2):227-233.
56. Luo Z Y W D, Meng W K (2018) Oral tranexamic acid is equivalent to topical tranexamic acid without drainage in primary total hip art hoursoplasty: A double-blind randomized clinical trial. Thrombosis Research 167:1-5.
57. Wei W B, Dang S J, Duan D P, & Wei L (2018) Comparison of intravenous and topical tranexamic acid in total knee art hoursoplasty. Bmc Musculoskel Dis 19(1):191.
58. Neeki M M D F, Toy J, Vaezazizi R, et al. (2017) Efficacy and Safety of Tranexamic Acid in Pre-hospital Traumatic Hemorrhagic Shock: Outcomes of the Cal-PAT Study. The Western Journal of Emergency Medicine 18(4):673-683.
59. Lipsky A M, et al. (2014) Tranexamic acid in the pre-hospital setting: Israel Defense Forces' initial experience. Injury 45(1):66-70.
60. Wu X W, Dubick M A, Schwacha M G, Cap A P, & Darlington D N (2017) Tranexamic Acid Attenuates the Loss of Lung Barrier Function in a Rat Model of Polytrauma and Hemorrhage with Resuscitation. Shock 47(4):500-505.
61. Wygrecka M, et al. (2009) Enolase-1 promotes plasminogen-mediated recruitment of monocytes to the acutely inflamed lung. Blood 113(22):5588-5598.
62. Lighvani S, et al. (2011) Regulation of macrophage migration by a novel plasminogen receptor Plg-R KT. Blood 118(20):5622-5630.
63. Gong Y, Hart E, Shchurin A, & Hoover-Plow J (2008) Inflammatory macrophage migration requires MMP-9 activation by plasminogen in mice. J Clin Invest 118(9):3012-3024.
64. Cap A P (2016) Plas min: a driver of hemovascular dysfunction. Blood 128(20):2375-2376.
65. Diebel L N, Martin J V, & Liberati D M (2017) Early tranexamic acid administration ameliorates the endotheliopathy of trauma and shock in an in vitro model. The journal of trauma and acute care surgery 82(6):1080-1086.
66. Diebel M E, Martin J V, Liberati D M, & Diebel L N (2018) The temporal response and mechanism of action of tranexamic acid in endothelial glycocalyx degradation. The journal of trauma and acute care surgery 84(1):75-80.
67. Hofman Z, de Maat S, Hack C E, & Maas C (2016) Bradykinin: Inflammatory Product of the Coagulation System. Clinical reviews in allergy & immunology 51(2):152-161.
68. Marcos-Contreras O A, et al. (2016) Hyperfibrinolysis increases blood-brain barrier permeability by a plas min- and bradykinin-dependent mechanism. Blood 128(20):2423-2434.
69. Halbgebauer R, et al. (2018) Hemorrhagic shock drives glycocalyx, barrier and organ dysfunction early after polytrauma. J Crit Care 44:229-237.
70. Gonzalez Rodriguez E, et al. (2017) Syndecan-1: A Quantitative Marker for the Endotheliopathy of Trauma. J Am Coll Surg 225(3):419-427.
71. Wu X, Darlington D N, & Cap A P (2016) Procoagulant and fibrinolytic activity after polytrauma in rat. American journal of physiology. Regulatory, integrative and comparative physiology 310(4):R323-329.
72. Darlington D N, et al. (2013) Acute coagulopathy of trauma in the rat. Shock 39(5):440-446.
73. Wu X, Dubick M A, Schwacha M G, Cap A P, & Darlington D N (2017) Tranexamic Acid Attenuates The Loss of Lung Barrier Function in a Rat Model of Polytrauma And Hemorrhage With Resuscitation. Shock 47(4):500-505.
74. Peng Z, et al. (2013) Fresh frozen plasma lessens pulmonary endothelial inflammation and hyperpermeability after hemorrhagic shock and is associated with loss of syndecan 1. Shock 40(3): 195-202.
75. Harrigan C, Lucas C E, & Ledgerwood A M (1983) Significance of Hypocalcemia Following Hypovolemic Shock. J Trauma 23(6):488-493.
76. Lucas C E, Sennish J C, Ledgerwood A M, & Harrigan C (1984) Parathyroid Response to Hypocalcemia after Treatment of Hemorrhagic-Shock. Surgery 96(4):711-716.
77. Denlinger J K, Na hourswold M L, Gibbs P S, & Lecky J H (1976) Hypocalcemia during Rapid Blood-Transfusion in Anesthetized Man. Brit J Anaesth 48(10):995-1000.
78. Giancarelli A, Birrer K L, Alban R F, Hobbs B P, & Liu-DeRyke X (2016) Hypocalcemia in trauma patients receiving massive transfusion. J Surg Res 202(1):182-187.
79. Webster S, Todd S, Redhead J, & Wright C (2016) Ionised calcium levels in major trauma patients who received blood in the Emergency Department. Emerg Med J 33(8):569-572.
80. Lier H, Krep H, Sc hoursoeder S, & Stuber F (2008) Preconditions of Hemostasis in Trauma: A Review. The Influence of Acidosis, Hypocalcemia, Anemia, and Hypothermia on Functional Hemostasis in Trauma. J Trauma 65(4):951-960.
81. Morotti A, et al. (2016) Association Between Serum Calcium Level and Extent of Bleeding in Patients With Intracerebral Hemorrhage. Jama Neurol 73(11):1285-1290.
82. Alberts M J & Sarode R (2016) Association Between Serum Calcium Level and the Size and Expansion of Intracerebral Hemorrhage. Jama Neurol 73(11):1276-1277.
83. Ho K M & Leonard A D (2011) Concentration-dependent effect of hypocalcaemia on mortality of patients with critical bleeding requiring massive transfusion: a cohort study. Anaesth Intens Care 39(1):46-54.
84. Duan C Y, Yang G M, Li T, & Liu L M (2015) Advances in Vascular Hyporeactivity after Shock: The Mechanisms and Managements. Shock 44(6):524-534.
85. Strawitz J G, Howard J M, & Artz C P (1955) Effect of Intravenous Calcium Gluconate on Post-Transfusion Hypotension—Clinical Observations. Arch Surg-Chicago 70(2):233-236.
86. Steele T, Kolamunnage-Dona R, Downey C, Toh C H, & Welters I (2013) Assessment and clinical course of hypocalcemia in critical illness. Crit Care 17(3).
87. Hastbacka J & Pettila V (2003) Prevalence and predictive value of ionized hypocalcemia among critically ill patients. Acta Anaesth Scand 47(10):1264-1269.
88. Egi M, et al. (2011) Ionized calcium concentration and outcome in critical illness. Crit Care Med 39(2):314-321.
89. Choi Y C & Hwang S Y (2008) The value of initial ionized calcium as a predictor of mortality and triage tool in adult trauma patients. J Korean Med Sci 23(4):700-705.
90. Xu J & Liu L (2005) The role of calcium desensitization in vascular hyporeactivity and its regulation after hemorrhagic shock in the rat. Shock 23(6):576-581.
91. Beauge L & Campos M A (1983) Calcium Inhibition of the Atpase and Phosphatase-Activities of (Na++K+)-Atpase. Biochim Biophys Acta 729(1):137-149.
92. Hock C E, Su J Y, & Lefer A M (1984) Salutary effects of nitrendipine, a new calcium entry blocker, in hemorrhagic shock. European journal of pharmacology 97(1-2):37-46.
93. Rose S, Pizanis A, & Silomon M (1997) Altered hepatocellular Ca²⁺ regulation during hemorrhagic shock and resuscitation. Hepatology 25(2):379-384.
94. Garcia-Dorado D, Ruiz-Meana M, Inserte J, Rodriguez-Sinovas A, & Piper H M (2012) Calcium-mediated cell death during myocardial reperfusion. Cardiovascular research 94(2):168-180.
95. Andreyev A T P, Rosenthal R E, Fiskum G (2018) Calcium uptake and cytoc hoursome c release from normal and ischemic brain mitochondria. Neurochemistry international 117:15-22.
96. Sperry J L, et al. (2018) Pre-hospital Plasma during Air Medical Transport in Trauma Patients at Risk for Hemorrhagic Shock. The New England journal of medicine 379(4):315-326.
97. Butler F K, Blackbourne L H, & Gross K (2015) The Combat Medic Aid Bag: 2025. CoTCCC Top 10 Recommended Battlefield Trauma Care Research, Development, and Evaluation Priorities for 2015. Journal of special operations medicine: a peer reviewed journal for SOF medical professionals 15(4):7-19.
98. Martini W Z, Cortez D S, & Dubick M A (2013) Comparisons of normal saline and lactated Ringer's resuscitation on hemodynamics, metabolic responses, and coagulation in pigs after severe hemorrhagic shock. Scandinavian journal of trauma, resuscitation and emergency medicine 21:86.
99. Butler F K, et al. (2014) Fluid Resuscitation for Hemorrhagic Shock in Tactical Combat Casualty Care: TCCC Guidelines Change 14-01-2 Jun. 2014. Journal of special operations medicine: a peer reviewed journal for SOF medical professionals 14(3):13-38.
100. Dart A B, Mutter T C, Ruth C A, & Taback S P (2010) Hydroxyethyl starch (HES) versus other fluid therapies: effects on kidney function. The Coc hoursane database of systematic reviews
   (1):CD007594.
101. Zarychanski R, et al. (2013) Association of hydroxyethyl starch administration with mortality and acute kidney injury in critically ill patients requiring volume resuscitation: a systematic review and meta-analysis. Jama 309(7):678-688.
102. Martini W Z, Dubick M A, & Blackbourne L H (2013) Comparisons of lactated Ringer's and Hextend resuscitation on hemodynamics and coagulation following femur injury and severe hemorrhage in pigs. The journal of trauma and acute care surgery 74(3):732-739; discussion 739-740.
103. Cordova C B, Cap A P, & Spinella P C (2014) Fresh whole blood transfusion for a combat casualty in austere combat environment. Journal of special operations medicine: a peer reviewed journal for SOF medical professionals 14(1):9-12.
104. Coc hoursane Injuries Group Albumin R (1998) Human albumin administration in critically ill patients: systematic review of randomised controlled trials. Bmj 317(7153):235-240.
105. Kheirabadi B S, Valdez-Delgado K K, Terrazas I B, Miranda N, & Dubick M A (2015) Is limited pre-hospital resuscitation with plasma more beneficial than using a synthetic colloid? An experimental study in rabbits with parenchymal bleeding. The journal of trauma and acute care surgery 78(4):752-759.
106. Kheirabadi B S, et al. (2016) Influences of limited resuscitation with plasma or plasma protein solutions on hemostasis and survival of rabbits with noncompressible hemorrhage. The journal of trauma and acute care surgery 81(1):42-49.
107. Jacob M, et al. (2008) Small-volume resuscitation with hyperoncotic albumin: a systematic review of randomized clinical trials. Crit Care 12(2):R34.
108. Studer N M, April M D, Bowling F, Danielson P D, & Cap A P (Albumin for Pre-hospital Fluid Resuscitation of Hemorrhagic Shock in Tactical Combat Casualty Care. Journal of special operations medicine: a peer reviewed journal for SOF medical professionals 17(2):82-88.
109. Darlington D N, et al. (2015) Trauma-Induced Coagulopathy Is Associated with a Complex Inflammatory Response in the Rat. Shock 44 Suppl 1:129-137.
110. Wu X, Schwacha M G, Dubick M A, Cap A P, & Darlington D N (2016) Trauma-Related Acute Lung Injury Develops Rapidly Irrespective of Resuscitation Strategy in the Rat. Shock 46(3 Suppl 1):108-114.
111. Chen J, et al. (2017) Limited Resuscitation With Fresh or Stored Whole Blood Corrects Cardiovascular and Metabolic Function in a Rat Model of Polytrauma and Hemorrhage. Shock 47(2):208-216.
112. Jastrow K M, et al. (2009) Early Cytokine Production Risk Stratifies Trauma Patients for Multiple Organ Failure. J Am Coll Surgeons 209(3):320-331.
113. Edens J W, et al. (2010) Predictors of Early Acute Lung Injury at a Combat Support Hospital: A Prospective Observational Study. J Trauma 69:S81-S86.
114. Heegard K D, et al. (2015) Early acute kidney injury in military casualties. J Trauma Acute Care 78(5):988-993.
115. Halbgebauer R, et al. (2018) Hemorrhagic shock drives glycocalyx, barrier and organ dysfunction early after polytrauma. J Crit Care 44:229-237.
116. Wutzler S, et al. (2011) Circulating Levels of Clara Cell Protein 16 But Not Surfactant Protein D Identify and Quantify Lung Damage in Patients With Multiple Injuries. J Trauma 71(2):E31-E36.
117. Akdemir H U, et al. (2014) The evaluation of different treatment protocols for trauma-induced lung injury in rats. J Thorac Dis 6(2):66-73.
118. Lin J L, Zhang W W, Wang L J, & Tian F (2018) Diagnostic and prognostic values of Club cell protein 16 (CC16) in critical care patients with acute respiratory distress syndrome. J Clin Lab Anal 32(2).
119. Moraes L, et al. (2018) Impact of Different Tidal Volume Levels at Low Mechanical Power on Ventilator-Induced Lung Injury in Rats. Front Physiol 9.
120. Bolignano D, et al. (2008) Neutrophil gelatinase-associated lipocalin (NGAL) as a marker of kidney damage. Am J Kidney Dis 52(3):595-605.
121. Stringham J R, et al. (2014) Mesenteric lymph diversion abrogates 5-lipoxygenase activation in the kidney following trauma and hemorrhagic shock. J Trauma Acute Care 76(5): 1214-1220.
122. Relja B, et al. (2010) Intestinal-FABP and Liver-FABP: Novel Markers for Severe Abdo minal Injury. Acad Emerg Med 17(7):729-735.
123. Szermutzky M, et al. (2010) I- and L-FABP are sensitive markers for abdo minal injuries in the early phase after multiple trauma but fail to indicate organ specific failure in the late phase after trauma. Inflamm Res 59:S46-S46.
124. van der Voort D, et al. (2005) Biosensors: basic features and application for fatty acid-binding protein, an early plasma marker of myocardial injury. Sensor Actuat B-Chem 105(1):50-59.
125. Pelsers MMAL, Hermens W T, & Glatz J F C (2005) Fatty acid-binding proteins as plasma markers of tissue injury. Clin Chim Acta 352(1-2):15-35.
126. Rodriguez E G, et al. (2017) Syndecan-1: A Quantitative Marker for the Endotheliopathy of Trauma. J Am Coll Surgeons 225(3):419-427.
127. Yuan X, et al. (2018) Targeting Hypoxia Signaling for Perioperative Organ Injury. Anesth Analg 126(1):308-321.
128. Debenham J S, et al. (2016) Discovery of N4Bis(4-methoxyphenyl)methyl1-4-hydroxy-2-(pyridazin-3-yl)pyrimidine-5-carboxami de (MK-8617), an Orally Active Pan-Inhibitor of Hypoxia-Inducible Factor Prolyl Hydroxylase 1-3 (HIF PHD1-3) for the Treatment of Anemia. Journal of medicinal chemistry 59(24):11039-11049.
129. Lee H B & Blaufox M D (1985) Blood volume in the rat. Journal of nuclear medicine: official publication, Society of Nuclear Medicine 26(1):72-76.
130. Singh P, Salih M, Leddy J J, & Tuana B S (2004) The muscle-specific calmodulin-dependent protein kinase assembles with the glycolytic enzyme complex at the sarcoplasmic reticulum and modulates the activity of glyceraldehyde-3-phosphate dehydrogenase in a Ca2+/calmodulin-dependent manner. J Biol Chem 279(34):35176-35182.
131. USFDA. Guidance for Industry: Estimating the Maximum Safe Starting Dose in Adult Healthy Volunteer. Rockville, MD: US Food and Drug Ad ministration; 2005.
132. Wu X et al. (2019) Effect of tranexamic acid administration on acute traumatic coagulopathy in rats with polytrauma and hemorrhage. PLoS One 3:14(10):e0223406.

1. A method for reducing organ failure and/or improving survivability in a patient with severe hemorrhage or hemorrhagic shock, comprising steps of:
a. administering resuscitation fluid through intravenous route, optionally administering an aliquot of 250 mL repeatedly up to 2000 mL while continuously monitoring a systolic blood pressure, radial pulse, and/or sensorium signs,
b. administering at least one composition comprising a therapeutically effective amount of anti-hemorrhage agent one time or multiple times, and
c. optionally administering vasopressors and/or inotropic agents;
wherein the resuscitation fluid comprises whole blood, plasma/red blood cells/platelets, crystalloid solution; colloid solution comprising human albumin, hydroxyl ethyl starch (HES), or dextran; hypertonic saline (5 ml/kg NaCl 7.5%) with or without dextran; or oxygen-carrying blood substitutes, or a combination thereof;
wherein the anti-hemorrhage agent comprises prolyl hydroxylase domain inhibitor (PHDi), antifibrinolytic agent, bradykinin receptor antagonist, or combination thereof that can be co-administered, and
wherein the vasopressor or inotropic is vasopressin, norepinephrine, epinephrine, dobutamine, or their synthetic equivalents;
wherein, the resuscitation fluid (i) is fresh whole blood or plasma/red blood cells/platelets, optionally at a ratio of about 1:about 1:about 1; or (ii) is a colloid solution comprising human albumin, and, optionally, wherein the resuscitation fluid further comprises fibrinogen.

2. (canceled)

3. (canceled)

4. (canceled)

5. The method of claim 1, wherein the PHDi is one selected from a group consisting of roxadustat (FG-4592), daprodustat (GSK-1278863), vadadustat (AKB-6548), molidustat (BAY 85-3934), enarodustat (JTZ-951), and MK-8617, and in particular MK-8617, wherein, optionally, MK-8617 is administered at a dose of 0.5-5 mg/kg, optionally 0.5-1.5 mg/kg; wherein, optionally, the composition comprising PHDi is administered is 5 min to 6 hr after onset of hemorrhage after onset of hemorrhage; and wherein, optionally, the composition comprising PHDi is administered is 5 min to 6 hr after onset of hemorrhage after administering the resuscitation fluid.

6. (canceled)

7. (canceled)

8. (canceled)

9. The method of claim 5, wherein the composition comprising PHDi is in a solid, semi-solid, or liquid dosage form, and administered orally or intravenously.

10. The method of claim 1, wherein the antifibrinolytic agent is one selected from a group consisting of tranexamic acid, aminocaproic acid, and aprotinin, and in particular tranexamic acid, wherein, optionally, tranexamic acid is administered at a dose of 5-50 mg/kg; wherein, optionally, the composition comprising antifibrinolytic agent is administered 5 min to 6 hr after onset of hemorrhage, and wherein, optionally, the composition comprising antifibrinolytic agent is administered 5 min to 6 hr after administering the resuscitation fluid.

11. (canceled)

12. (canceled)

13. (canceled)

14. The method of claim 10, wherein the composition comprising antifibrinolytic agent is in a dosage form of solid, semi-solid, or liquid dosage form, and administered through oral, parenteral, or topical route.

15. The method of claim 1, wherein the bradykinin receptor antagonist is a bradykinin B2 receptor antagonist, icatibant; wherein, optionally, icatibant is administered at a dose of 0.1-1.0 mg/kg; wherein, optionally, the composition comprising bradykinin receptor antagonist is administered 5 min to 6 hr after onset of hemorrhage; and wherein, optionally, the composition comprising bradykinin receptor antagonist is administered 5 min to 6 hr after administering the resuscitation fluid.

16. (canceled)

17. (canceled)

18. (canceled)

19. The method of claim 15, wherein the composition comprising bradykinin receptor antagonist is in a dosage form of solid, semi-solid, or liquid dosage form, and administered through oral, parenteral, or topical route.

20. The method of claim 1, wherein the composition further comprises pharmaceutically suitable excipients.

21. The method of claim 1, wherein the method further comprises physically controlling the hemorrhage and/or securing airway and breathing,

22. The method of claim 1, wherein the anti-hemorrhage agent or combination thereof are co-administered with calcium supplement, wherein the calcium supplement is calcium gluconate or calcium chloride,

23. The method of claim 21, wherein the calcium supplement is 10% calcium gluconate or calcium chloride and administered at a dose of 0.01-1 ml/kg, in particular 0.1 ml/kg.

24. The method of claim 21, wherein the calcium supplement is in a solid- or liquid dosage form for oral or parenteral administration.

25. A kit for administration of a composition/compositions to reduce organ failure and/or improve survivability in a patient with severe hemorrhage or hemorrhagic shock, comprising;
a. at least one composition comprising a therapeutically effective amount of anti-hemorrhage agent for one time- or multiple time administration, wherein the composition is in a liquid dosage form contained in an injectable container of 5-25 mL volume or solid, semi-solid, or liquid dosage form for oral administration,
b. optionally comprising non-flammable hand sanitizer, at least one pair of disposable nonlatex gloves, sterile saline for wound washing, and sterilized cleaning pads,
wherein the anti-hemorrhage agent comprises prolyl hydroxylase domain inhibitor (PHDi), antifibrinolytic agent, bradykinin receptor antagonist, or combination thereof that can be co-administered, and wherein the composition may further comprise pharmaceutically suitable excipients in addition to the active ingredient.

26. The kit of claim 25, wherein the kit further comprises colloid solution of 5-50% human albumin, in particular 25% human albumin, and wherein it may further comprise fibrinogen.

27. The kit of claim 25, wherein the PHDi is one selected from a group consisting of roxadustat (FG-4592), daprodustat (GSK-1278863), vadadustat (AKB-6548), molidustat (BAY 85-3934), enarodustat (JTZ-951), and MK-8617, and in particular MK-8617.

28. The kit of claim 25, wherein the antifibrinolytic agent is one selected from a group consisting of tranexamic acid, aminocaproic acid, and aprotinin, and in particular tranexamic acid.

29. The kit of claim 25, wherein the bradykinin receptor antagonist is a bradykinin B2 receptor antagonist, icatibant.

30. The kit of claim 25, wherein the kit further comprises calcium gluconate or calcium chloride.
